(19) United States
(12) Patent Application Publication
(10) Pub. No.: US 2025/0248622 A1
(43) Pub. Date: Aug. 7, 2025

(54) Systems, methods and devices for assessing hearing

(71) Applicant: THE BIONICS INSTITUTE OF AUSTRALIA, East Melbourne (AU)

(72) Inventors: Onn Wah Lee, East Melbourne (AU); Colette McKay, East Melbourne (AU); Gautam Balasubramanian, East Melbourne (AU); Darren Mao, East Melbourne (AU); Julia Wunderlich, East Melbourne (AU)

(21) Appl. No.: 18/856,968

(22) PCT Filed: Apr. 14, 2023
(86) PCT No.: PCT/AU2023/050308

§ 371 Date: Oct. 15, 2024

(30) Foreign Application Priority Data
Apr. 14, 2022 (AU) 2022901011

(51) Int. Cl. A61B 5/12; A61B 5/00; A61B 5/0205

(52) U.S. Cl. CPC A61B 5/125; A61B 5/0075; A61B 5/0205; A61B 5/7235

(57) ABSTRACT

Described embodiments generally relate to a method of assessing the hearing of a subject. The method comprises presenting a first aural stimulus to the subject; presenting a second aural stimulus to the subject, the second aural stimulus being different to the first aural stimulus; receiving at least one physiological response signal relating to the second aural stimulus received by the subject; comparing at least one parameter related to the at least one physiological response signal with a control parameter; and determining an auditory discrimination response of the subject based on the outcome of the comparison.

### TECHNICAL FIELD

Embodiments generally relate to systems, methods and devices for assessing hearing. In particular, described embodiments are directed to systems, methods and devices for assessing hearing by assessing sound detection and sound discrimination ability.

### BACKGROUND

Accurate assessment of hearing is important for screening and diagnosis of hearing impairment and also for validation of hearing instrument fitting. As well as testing for whether a patient can hear certain sounds, it can be important to test for whether a patient can discriminate between different sounds, particularly speech sounds. Speech discrimination is one of the four basic types of speech perception that contributes to understanding conversational speech.

Hearing assessments to determine the ability of patients to perceive and discriminate speech sounds are challenging, and are normally determined using task-based behavioural tests, if at all. The subject would need to understand the task, pay attention to the stimuli, give verbal or non-verbal responses, and react within a time window. However, some patients, such as infants that haven't yet developed language and have short attention spans, may find these tasks difficult. This means that the ability to detect or discriminate between sounds might only be able to be tested once a child develops language. However, this delay in information can lead to serious impacts on language development.

It is desired to address or ameliorate one or more shortcomings or disadvantages associated with prior systems for hearing assessment, or to at least provide a useful alternative thereto.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Some embodiments relate to a method of assessing the hearing of a subject, the method comprising:
presenting a first aural stimulus to the subject;
presenting a second aural stimulus to the subject, the second aural stimulus being different to the first aural stimulus;
receiving at least one physiological response signal relating to the second aural stimulus received by the subject;
comparing at least one parameter related to the at least one physiological response signal with a control parameter; and
determining an auditory discrimination response of the subject based on the outcome of the comparison.

In some embodiments, the first aural stimulus and the second aural stimulus are presented consecutively without any silence interval between the first aural stimulus and the second aural stimulus.

Some embodiments further comprise modelling the physiological response signal to create a response model.

In some embodiments, the model captures unique statistical properties in the neural response data pertaining to its neighbourhood covariance structure relative to aural stimulation onset, spanning the length of the expected response and across one or more responses.

According to some embodiments, the model is generated using a stochastic process.

In some embodiments, the response model captures at least two concurrent neural responses in response to one aural stimulus.

In some embodiments, the at least one of the neural processes relates to activation of the cortical auditory system.

Some embodiments further comprise using the modelled neural response data relating to the activation of the cortical auditory system to determine a measure corresponding to whether or not the subject discriminated between the first aural stimulus and the second aural stimulus.

Some embodiments further comprise using the modelled neural response data relating to the activation of the cortical auditory system to determine a measure corresponding to whether or not the subject detected the first stimulus or the second stimulus.

According to some embodiments, at least one of the neural processes relates to activation of the brain arousal system.

Some embodiments further comprise using the modelled neural response data relating to the activation of the brain arousal system to determine a measure corresponding to a difficulty that the subject experienced in discriminating between the first aural stimulus and the second aural stimulus.

Some embodiments further comprise using the modelled neural response data relating to the activation of the brain arousal system to determine a measure corresponding to whether or not the subject detected the stimulus (Response 1290 in FIG. 12B).

In some embodiments, the control parameter is determined based on a model of a baseline response signal.

According to some embodiments, the baseline response signal comprises a physiological response signal measured during a time not aligned with a time when the second aural stimulus is being delivered to the subject.

In some embodiments, the baseline response signal comprises a physiological response signal measured during a time when the first aural stimulus is being delivered to the subject.

In some embodiments, the baseline response signal comprises a physiological response signal measured during a time when no stimulus is being delivered to the subject.

According to some embodiments, comparing at least one parameter related to the at least one physiological response signal with a control parameter comprises using test statistics to compare whether the response model and the model of the baseline response signal are different.

In some embodiments, the data used to generate the models are permuted and create a collection of statistical measures.

According to some embodiments, the collection of statistical measures is used to generate a confidence level for the response detection decision.

According to some embodiments, the first aural stimulus comprises a repeating speech syllable presented as a habituation stimulus.

In some embodiments, the second aural stimulus comprises a repeating speech syllable presented as a dishabituation stimulus.

According to some embodiments, the physiological response signal comprises fNIRS data generated by at least one optode located on a scalp of the subject.

In some embodiments, the fNIRS data comprises neural response data.

In some embodiments, the fNIRS data comprises cardiac data.

According to some embodiments, the physiological response signal comprises cardiac data generated by a cardiac monitor.

In some embodiments, the cardiac data comprises at least one of a heart rate, heart rate variability, blood pressure and/or breathing rate.

According to some embodiments, the first aural stimulus and the second aural stimulus are selected to create a speech contrast, the speech contrast being the result of a difference in one or more speech features between the first aural stimulus and the second aural stimulus for which a discrimination assessment is desired.

In some embodiments, the speech contrast comprises at least one of a vowel contrast, consonant contrast, vowel difference cue, or a difference in place of consonant articulation.

Some embodiments further comprise determining an auditory discrimination response of the subject based on the outcome of the comparison.

In some embodiments, the first aural stimulus is presented for a duration of between 1 and 35 seconds.

In some embodiments, the first aural stimulus is presented for a duration of between 9 and 14 seconds.

In some embodiments, the second aural stimulus is presented for a duration of between 1 and 35 seconds.

In some embodiments, the second aural stimulus is presented for a duration of less than seconds.

Some embodiments further comprise repeating the steps of presenting the first aural stimulus, presenting a second aural stimulus and receiving at least one physiological response signal.

In some embodiments, the steps of presenting the first aural stimulus, presenting a second aural stimulus and receiving at least one physiological response signal are repeated until a stopping criterion is met.

According to some embodiments, the collection of statistical measures is used to calculate a stopping criterion. Some embodiments further comprise:
combining the received physiological response signals into a combined single physiological response signal, and
combining at least two physiological response signals into a combined single baseline response signal,
wherein comparing the at least one parameter related to the at least one physiological response signal with a control parameter comprises comparing the combined single physiological response signal with the combined single baseline response signal.

In some embodiments, combining at least two physiological response signals into a combined single baseline response signal comprises combining the at least two physiological response signals measured during a time not aligned with a time when the second aural stimulus is being delivered to the subject.

In some embodiments, combining at least two physiological response signals into a combined single baseline response signal comprises combining the at least two physiological response signals measured during a time when no stimulus is being delivered to the subject.

In some embodiments, combining at least two physiological response signals into a combined single baseline response signal comprises combining the at least two physiological response signals measured during a time when the first aural stimulus is being delivered to the subject.

Some embodiments further comprise communicating the outcome to a user via a user interface.

Some embodiments further comprise communicating the outcome to an external computing system.

Some embodiments relate to a system for assessing the hearing of a subject, the system comprising:
at least one stimulation delivery member;
at least one physiological signal sensor;
memory storing executable code; and
a processor configured to access and execute the code stored in the memory;
wherein, when executing the code, the processor is caused to:
   present, via the at least one stimulation delivery member, a first aural stimulus to the subject;
   present, via the at least one stimulation delivery member, a second aural stimulus to the subject, the second aural stimulus being different to the first aural stimulus;
   receive, from the at least one physiological signal sensor, at least one physiological response signal relating to the second aural stimulus received by the subject;
   compare at least one parameter related to the at least one physiological response signal with a control parameter; and
   determine an auditory discrimination response of the subject based on the outcome of the comparison.

Some embodiments relate to a method of assessing the hearing of a subject, the method comprising:
presenting an aural stimulus to the subject;
receiving at least one physiological response signal relating to the aural stimulus received by the subject;
modelling the physiological response signal to create a response model, wherein the response model captures at least two concurrent neural responses in response to one aural stimulus;
using the modelled neural response data to determine a measure corresponding to whether or not the subject detected the aural stimulus.

In some embodiments, the at least one of the neural processes relates to activation of the cortical auditory system.

In some embodiments, at least one of the neural processes relates to activation of the brain arousal system.

### BRIEF DESCRIPTION OF DRAWINGS

Embodiments are described in further detail below, by way of example and with reference to the accompanying drawings, in which:

FIG. 1 shows a block diagram of a hearing assessment system according to some embodiments;

FIG. 2 shows a diagram of optodes from the system of FIG. 1 being used to perform hearing assessment on a patient;

FIG. 3 shows a flow diagram illustrating an example method using the system of FIG. 1;

FIG. 4 shows a flow diagram illustrating an example first stimulus delivery method using the system of FIG. 1;

FIG. 5A shows a flow diagram illustrating an example habituation portion of a second stimulus delivery method using the system of FIG. 1;

FIG. 5B shows a flow diagram illustrating an example dishabituation portion of a second stimulus delivery method using the system of FIG. 1;

FIG. 6 shows a flow diagram illustrating an example first response feature extraction method using the system of FIG. 1;

FIG. 7 shows a flow diagram illustrating an example second response feature extraction method using the system of FIG. 1;

FIG. 8 shows a timing diagram of a first stimulation protocol that may be provided by the system of FIG. 1 performing the method of FIG. 4;

FIG. 9 shows a graph of heart rate response signals measured for a subject in response to the subject being presented the stimulation protocol of FIG. 8;

FIGS. 10A and 10B show graphs of hemodynamic response signals measured for a subject in response to the subject being presented the stimulation protocol of FIG. 8;

FIG. 11 shows a timing diagram of a second stimulation protocol that may be provided by the system of FIG. 1 performing the method of FIG. 5;

FIGS. 12A and 12B show graphs of hemodynamic response signals measured for a subject in response to the subject being presented a first phase of the stimulation protocol of FIG. 11;

FIGS. 13A and 13B show graphs of hemodynamic response signals measured for a subject in response to the subject being presented a second phase of the stimulation protocol of FIG. 11;

FIG. 14 shows a graph of heart rate response signals measured for a subject in response to the subject being presented the second phase of the stimulation protocol of FIG. 11; and

FIG. 15 shows a graph illustrating an example of a stopping criterion, which allows adaptive stopping of stimulus presentation to optimize experiment duration.

### DETAILED DESCRIPTION

Embodiments generally relate to systems, methods and devices for assessing hearing. In particular, described embodiments are directed to systems, methods and devices for assessing hearing by assessing sound discrimination ability.

Hearing assessments to determine sound discrimination ability are challenging to perform, especially on young infants. As a result, these tests are rarely conducted, meaning that a child's inability to discern different types of sound may not be recognised until that child develops language, or when the child has a developmental delay in their language skills.

Functional near-infrared spectroscopy (fNIRS) is a child-friendly and objective neuroimaging tool. fNIRS uses light in the near-infrared spectrum to evaluate neural activity in the brain via changes in blood oxygenation. This is possible due to a range of wavelengths of near-infrared light over which skin, tissue, and bone are mostly transparent but in which blood is a stronger absorber of the light. Differences in the light absorption levels of oxygenated and deoxygenated blood allow the measurement of relative changes in blood oxygenation in response to brain activity.

fNIRS raw data measures changes in blood oxygenation, from which neural activity can be extracted using a series of processing steps. As well as neural activity, fNIRS data consists of systemic information such as biosignal or physiological information. Biosignal or physiological information signals can be extracted from the fNIRS raw data. Biosignal or physiological information in this context may include cardiac information, respiratory information and Mayer wave information, and may include information such as heart rate, heart beat pulses, breathing and blood pressure changes. These biosignal or physiological information signals are often separated and rejected in fNIRS analyses, in order to avoid these additional signals from interfering with the measurement of relative changes in blood oxygenation in response to brain activity. However, this biosignal or physiological information can be used to measure responses to stimuli in patients. Alternatively or additionally to fNIRS extracted biosignal or physiological data, biosignal or physiological data measured using different sensor types may also be used to measure responses to stimuli in patients.

Patient responses as measured using fNIRS data and other sensor data are sensitive to states of alertness and vigilance in the subject. For example, a subject's stimuli-evoked heart rate change response as measured using fNIRS data reduces in magnitude when a subject listens to the same stimuli repeatedly. This is known as the habituation effect, which also occurs when the patient is asleep. A renewal of stimuli-evoked responses can be observed in fNIRS data for subjects following the presentation of novel stimuli.

This is known as the dishabituation effect. fNIRS data that indicates the presence of the dishabituation effect can be used to measure the discrimination of speech sounds in a subject. Described embodiments relate to systems, methods and devices for extracting response signals to objectively measure speech discrimination.

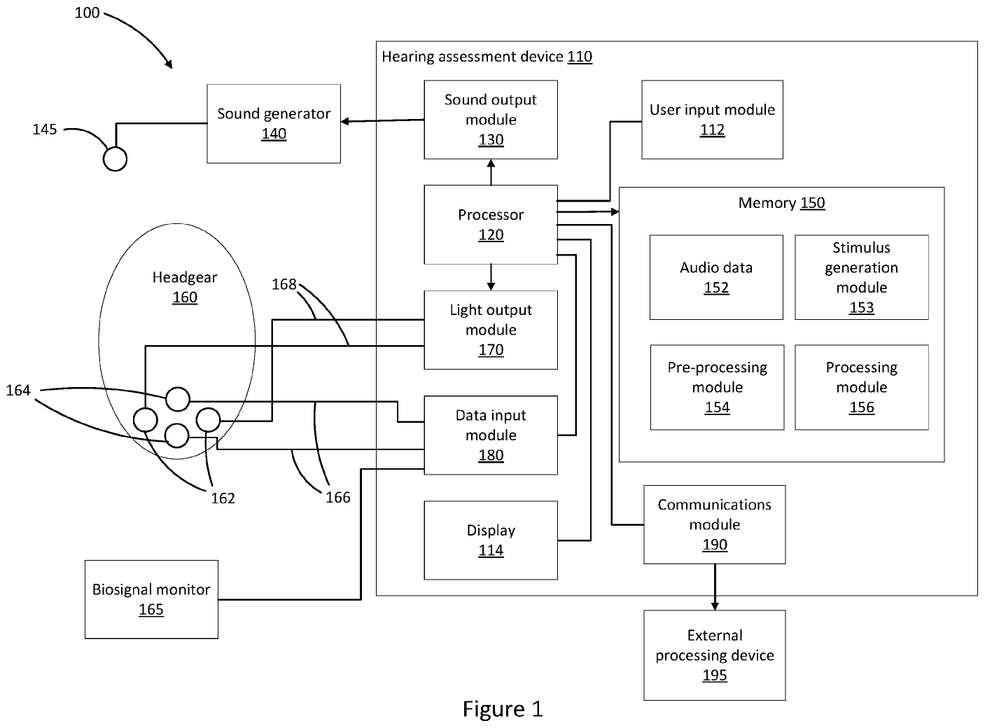

FIG. 1 shows a system 100 for hearing assessment. System 100 may use fNIRS data to assess neural and other biosignal response signals evoked by a patient in response to an auditory stimulation signal. According to some embodiments, system 100 may additionally or alternatively use biosignal data to assess biosignal response signals evoked by a patient in response to an auditory stimulation signal. In some embodiments, system 100 may filter fNIRS data to remove some biosignal information signals, such as cardiac information signals and/or respiratory information signals, for example. According to some alternative embodiments, system 100 may use the biosignal information signals extracted from the fNIRS data as additional or alternative sources of data for the hearing assessment.

System 100 comprises a hearing assessment device 110. In the illustrated embodiment, system 100 further comprises a sound generator 140 in communication with the hearing assessment device 110, and a stimulation member 145 in communication with sound generator 140. The illustrated embodiment also shows an external processing device 195 in communication with hearing assessment device 110. According to some embodiments, system 100 also comprises headgear 160. According to some embodiments, system 100 also comprises a biosignal monitor 165. According to some embodiments, system 100 may comprise only one of headgear 160 and biosignal monitor 165. According to some embodiments, system 100 may comprise both headgear 160 and biosignal monitor 165.

Hearing assessment device 110 has a processor 120, which communicates with a sound output module 130, memory 150, a light output module 170, a data input module 180 and a communications module 190. In the illustrated embodiment, sound generator 140 is a separate unit from assessment device 110. However, in some embodiments, sound generator 140 may be part of hearing assessment device 110. Sound generator 140 may be configured to receive sound output data from sound output module 130.

Stimulation member 145 may be a speaker, earphone, hearing aid, hearing instrument, implantable auditory prosthesis comprising implantable electrodes, transducer, cochlear implant, brain stem implant, auditory midbrain implant, or other component used to provide aural stimulation to a patient. According to some embodiments, the aural stimulation may be delivered through bone conduction. According to some embodiments, two stimulation members 145 may be used, to provide binaural stimulation. According to some embodiments, stimulation member 145 may be an audiometric insert earphone, such as the ER-3A insert earphone by E-A-RTONE™ 165 GOLD, US, or a speaker, such as the Genelec 8020B Studio Monitor. In some embodiments, stimulation member 145 may interface with another component, such as a hearing aid or cochlear implant, in order to provide aural stimulation to the patient. Sound generator 140 communicates with stimulation member 145 to cause the stimulation member 145 to produce a range of aural stimulation signals to assess the patient's hearing. According to some embodiments, sound generator 140 may communicate sounds output data to stimulation member 145. According to some embodiments, sound generator 140 causes the stimulation member 145 to play back previously recorded audio. When the patient has a cochlear implant, stimulation member 145 may be a computer and pod that interfaces directly with a coil of the cochlear implant, to cause the implant to produce electrical pulses that evoke sound sensations. In this case, sound generator 140 generates and transmits instructions for the patterns of electrical pulses to stimulation member 145.

Headgear 160 includes a number of optodes 162/164, having at least one source optode 162 and at least one detector optode 164. Source optodes 162 are configured to receive signals via transmission channels 168, and detector optodes 164 are configured to provide output signals via measurement channels 166. Headgear 160 may be a cap, headband, or other head piece suitable for holding optodes 162/164 in position on a patient's head. Optodes 162/164 may be arranged on headgear 160 to be positioned in the region of the auditory cortex of the patient when headgear 160 is worn correctly. In some cases, headgear 160 may have between 1 and 32 source optodes 162 and between 1 and 32 detector optodes 164. Source optodes 162 and their paired detector optodes 164 may be spaced at between 0.5 and 5 cm from one another on headgear 160. In some embodiments, headgear 160 may be an Easycap 32 channel standard EEG recording cap, and optodes 162/164 may be attached using rivets or grommets. According to some embodiments, headgear 160 may be an NIRScout system NIRScap by NIRx Medical technologies LLC, Germany. In some embodiments, headgear 160 may have 16 source optodes 162 and 16 detector optodes 164. According to some embodiments, headgear 160 may be a 64 channel continuous wave device.

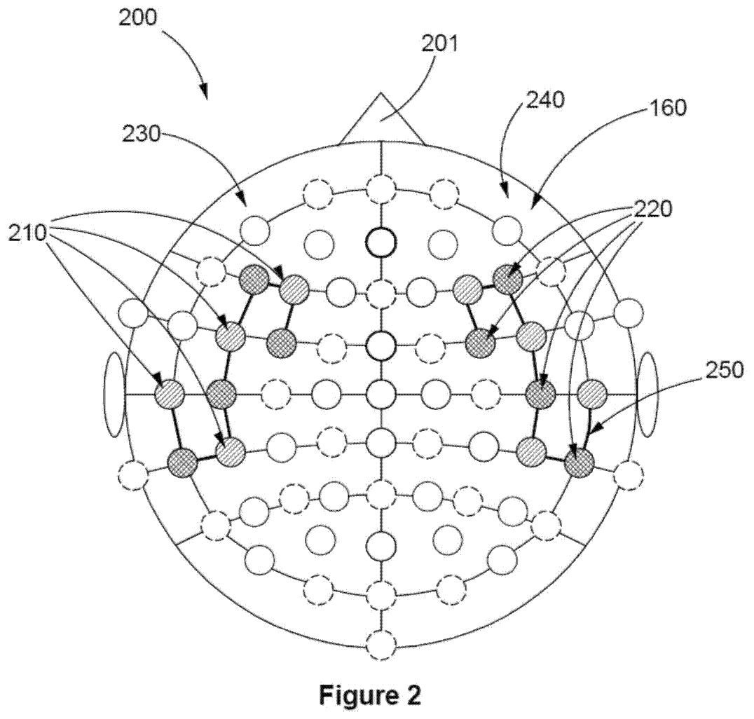

According to some embodiments, headgear 160 may be arranged as per the diagram shown in FIG. 2, and as described in further detail below.

Referring again to system 100 of FIG. 1, biosignal monitor 165 may comprise one or more devices configured to measure biosignal information of a patient. The biosignal information may include cardiac information such as heartbeat, systemic blood pressure or heart rate; respiration information such as respiration rhythm; and Mayer waves. Biosignal monitor 165 may comprise one or more of a heart rate monitor, a respiratory monitor, blood pressure monitor and Mayer wave monitor, for example.

Although only one external processing device 195 is shown, assessment device 110 may be in communication with more than one external processing device 195, which may in some embodiments be desktop or laptop computers, mobile or handheld computing devices, servers, distributed server networks, or other processing devices. According to some embodiments, external processing device 195 may be running a data processing application such as Matlab 2016b (Mathworks, USA), for example. In some embodiments, external processing device 195 may be running a Python based data processing application. In some alternative embodiments, hearing assessment device 110 may not be in communication with any external processing devices.

Processor 120 may include one or more data processors for executing instructions, and may include one or more of a microprocessor, microcontroller-based platform, a suitable integrated circuit, and one or more application-specific integrated circuits (ASIC's).

Sound output module 130 is arranged to receive instructions from processor 120 and send signals to sound generator 140, causing sound generator 140 to provide signals to stimulation member 145. Where stimulation member 145 comprises a speaker or earphone, the signals may include an acoustic signal delivered via the earphone or speaker in the sound field. Where stimulation member 145 comprises a hearing instrument, the signals may comprise a digital sound file delivered via direct audio input to the hearing instrument. Where stimulation member 145 comprises an implantable auditory prosthesis, the signals may comprise instructions for an electrical signal to be delivered by implanted electrodes in the implantable auditory prostheses.

Memory 150 may include one or more memory storage locations, either internal or external to optical read system 100, and may be in the form of ROM, RAM, flash or other memory types. Memory 150 is arranged to be accessible to processor 120, and to store data that can be read and written to by processor 120. For example, memory 150 may store audio data 152, which may comprise stimulus audio to be delivered to a subject via system 100. Memory 150 may also contain program code that is executable by processor 120, in the form of executable code modules. These may include stimulus generation module 153, pre-processing module 154, and processing module 156.

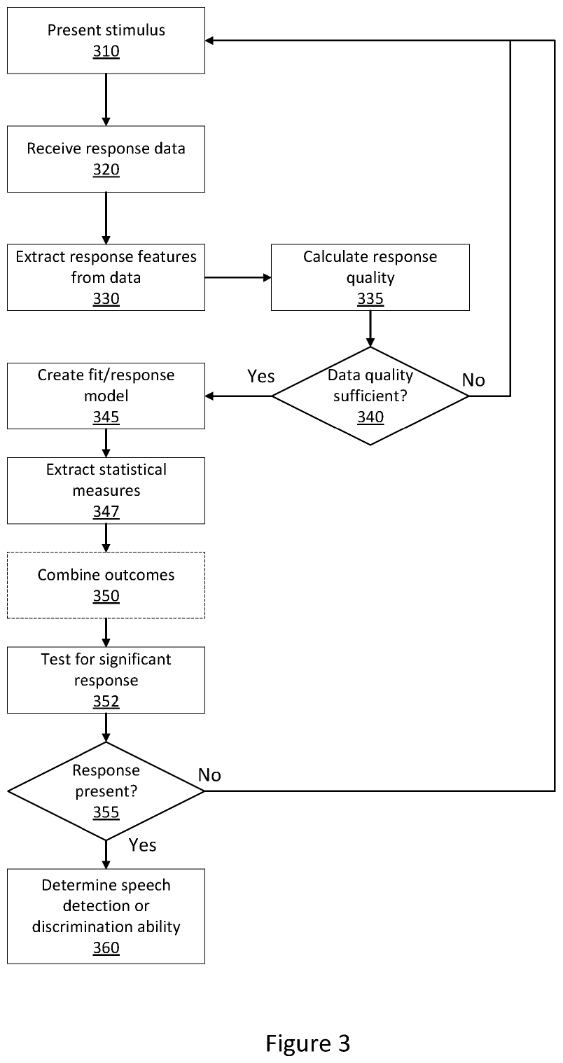

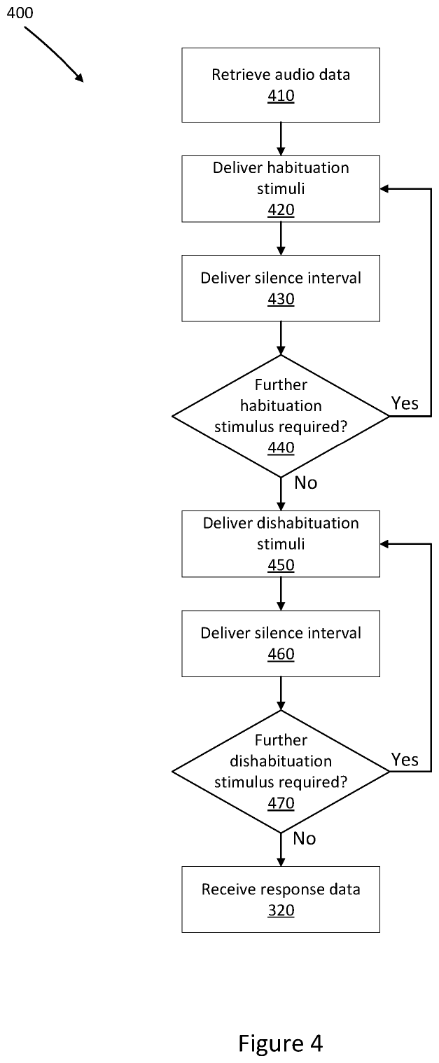

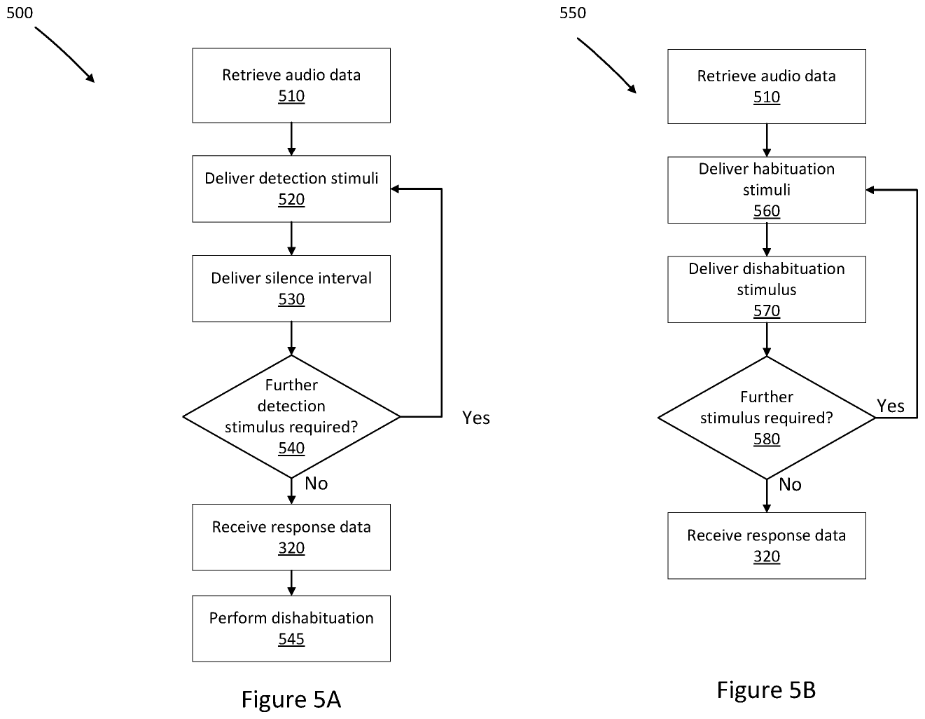

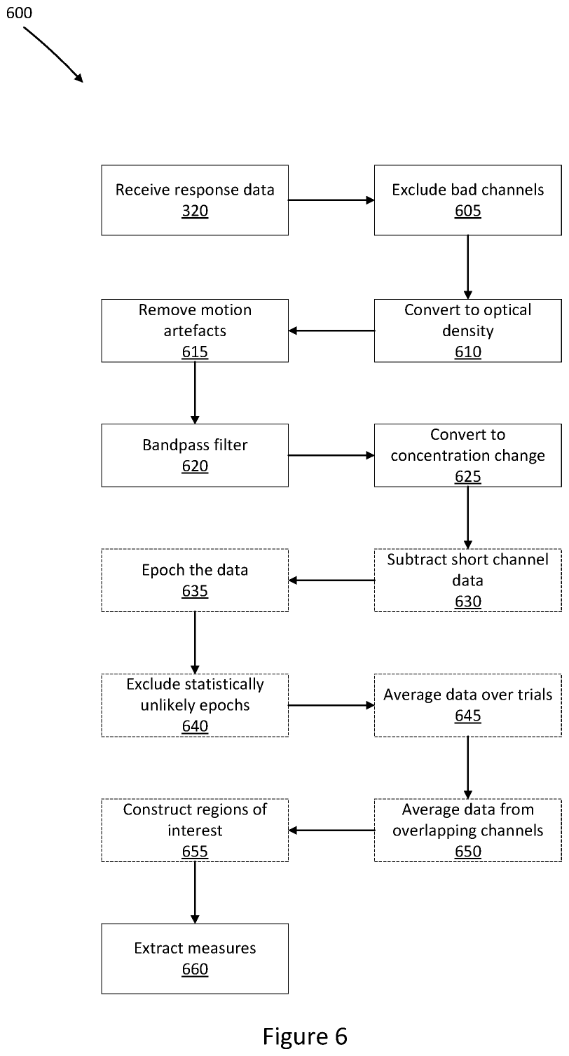

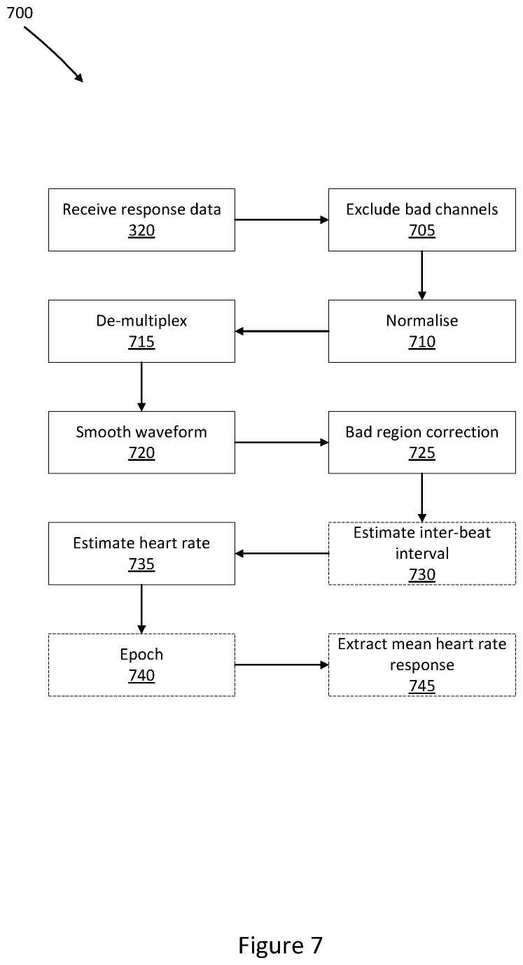

According to some embodiments, processor 120 may execute stimulus generation module 153 to perform the method of at least one of FIGS. 4, 5A and 5B, as described in further detail below. This may cause processor 120 to generate auditory stimulus data such as an audio track to be delivered to a subject via stimulation member 145. In some embodiments, processor 120 may execute pre-processing module 154 to perform the methods of one of FIGS. 6 and 7, as described in further detail below. This may cause processor 120 to perform pre-processing on data received from data input module 180, to extract certain response features from the data. According to some embodiments, processor 120 may execute processing module 156 to perform the method of FIG. 3, as described in further detail below. This may cause processor 120 to perform processing of the pre-processed data generated by pre-processing module 154, to determine whether a subject exhibited a discrimination response based on the provided stimulus.

Light output module 170 is configured to receive instructions from processor 120 and send signals to source optodes 162 via transmission channels 168, causing source optodes 162 to generate near infra-red light. Data input module 180 is configured to receive data signals from detector optodes 164 via measurement channels 166, the data signals being generated based on the near infra-red light detected by detector optodes 164. According to some embodiments, data input module 180 may comprise NIRStar_v15.3 acquisition software, for example.

Communications module 190 may allow for wired or wireless communication between assessment device 110 and external processing device 195, and may utilise Wi-Fi, USB, Bluetooth, or other communications protocols.

User input module 112 may be configured to accept input from a number of user input sources, such as a touchscreen, keyboard, buttons, switches, electronic mice, and other user input controls. User input module 112 is arranged to send signals corresponding to the user input to processor 120. Display 114 may include one or more screens, which may be LCD or LED screen displays in some embodiments, and be caused to display data on the screens based on instructions received from processor 120. In some embodiments, assessment device 110 may further include lights, speakers, or other output devices configured to communicate information to a user.

Sound output module 130 may communicate with sound generator 140, to cause sound generator 140 to generate a sound or audio signal based on the instructions received from sound output module 130. Sound generator 140 may output the sound signal to stimulation member 145 to cause stimulation member 145 to produce one or more sounds. According to some embodiments, sound output module 130 may comprise Presentation software by Neurobehavioral Systems, Inc., and may be configured to pass on sound or audio data as retrieved from audio data 152.

According to some embodiments, audio data 152 may comprise speech stimuli narrated by a human speaker, which may comprise syllables and/or words. For example, the speech stimuli may comprise consonant-vowel speech syllables in some embodiments. According to some embodiments, the audio data 152 may be pre-recorded using a microphone, such as an AT2020USB+microphone, for example. The pre-recorded audio may be sampled at 44.1 kHz and 16 bits resolution, in some embodiments.

According to some embodiments, the audio data 152 may comprise stimulus blocks comprising multiple repeating instances of speech, such as repeating instances of spoken syllables or words. For example, pre-recorded speech syllables may be pre-processed and repeated to form a stimulus block, then stored to audio data 152. According to some embodiments, the pre-processing may comprise passing the recording through a high pass filter to remove low frequency noise. For example, the audio may be high-pass filtered with a cut-off at 20 Hz and 6 dB roll-off per octave. According to some embodiments, the pre-processing may further comprise trimming the audio. For example, the audio may be trimmed down to 500 milliseconds. The pre-processed audio may then be repeated to form a stimulus block, and stored to audio data 152. For example, the pre-processed audio may be repeated ten times with no interstimulus silence interval to form a five-second stimulus block, in some embodiments. Where multiple stimulus blocks comprising different speech stimuli audio recordings are used, the intensity of each stimulus block may be equalized using the root-mean-square method. According to some embodiments, each stimulus block may further be calibrated to 65 dB SPL.

According to some embodiments, multiple stimulus blocks may be stored together within a single audio track within audio data 152. For example, an audio track may include a first stimulus block comprising a first repeated speech stimulus, and a second stimulus block comprising a second repeated speech stimulus. According to some embodiments, the speech stimuli for the stimulus blocks within an audio track may be selected to create speech contrasts. In some embodiments, these speech contrasts differ in their spectral content and/or have temporal differences. For example, the differences may include one or more of vowel difference cues, consonants with differing places of articulation, vowel contrasts or consonant contrasts. For example, the speech syllable pair “baa”/“tea” contains vowel difference cue and consonants /b/ and /t/ that are differed in place of articulation. The speech syllable pair “boo”/“bee” has a vowel contrast, which differs in broad spectral profile, especially in the first and second formant spectral power. The speech syllable pair “she”/“see” differs only in consonant place of articulation of /f/ and /s/ sound, requiring higher spectral discrimination ability.

According to some embodiments, processor 120 may execute stimulus generation module 153 to generate audio tracks by combining speech stimuli recordings into stimulus blocks for storage in audio data 152. According to some embodiments, the audio tracks may be generated ahead of time and stored for retrieval and playback at the time that stimulation is delivered. In some embodiments, the audio tracks may be generated in real time as they are delivered to the subject.

Stimulation member 145 may be positioned in, on or near a patient, in order to aurally stimulate the patient. Where headgear 160 is being used, headgear 160 may be positioned on the patient so that optodes 162/164 are positioned in proximity to the temporal lobe of the patient. Where biosignal monitor 165 is being used, biosignal monitor 165 may be positioned to measure biosignal information of the patient, such as heartbeat or respiratory information, for example. When the patient hears a sound due to the stimulation provided by stimulation member 145, the neural activity in the patient's brain in the measured area, which may be at or around the auditory cortex, changes. According to some embodiments, the patient's heart rate, heart rate variability, blood pressure and/or breathing rate may also increase or decrease when the patient hears a sound. Optodes 162/164 are used to measure the changes in blood oxygenation in the auditory cortex region, which may be a result of changes in neural activity, and/or changes in heart rate, heart rate variability, blood pressure and/or breathing. Processor 120 sends instructions to light output module 170, which controls the light emitted by source optodes 162 by sending signals along transmission channels 168. This light passes through the measured region of the patient's brain, and some of the light is reflected back to detector optodes 164.

Data collected by detector optodes 164 is carried by measurement channels 166 to data input module 180, which communicates with processor 120. Biosignal monitor 165 may also be used to measure changes in heart rate, heart rate variability, blood pressure and/or breathing, and data signals collected by biosignal monitor 165 may also be carried by measurement channels to data input module 180, which communicates with processor 120. In some cases, the data may be stored in memory 150 for future processing by assessment device 110 or external computing device 195. In some embodiments, the data may be processed by assessment device 110 in real time. Processor 120 may execute pre-processing module 154 to pre-process the data as it is captured.

Processor 120 executing pre-processing module 154 may be caused to process the data by removing noise, and unwanted signal elements. According to some embodiments, these may include signal elements such as those caused by breathing of the patient, the heartbeat of the patient, a Mayer wave, a motion artefact, non-hearing related brain activity, and the data collection apparatus, such as measurement noise generated by the hardware. In some embodiments, the signal elements caused by some physiological processes, such as breathing or heartbeats, may be kept for further analysis, as described below. According to some embodiments, pre-processing module 154 may comprise a custom script in MATLAB and the NIRS Brain AnalyzIR Toolbox. The effects of processor 120 executing pre-processing module 154 are described below in further detail below with reference to FIGS. 6 and 7.

As described in further detail below with reference to FIG. 3, processor 120 may subsequently execute processing module 156, which may determine whether a subject has exhibited a discrimination response in response to the presented auditory stimulation. According to some embodiments, this may include processor 120 determining whether certain dishabituation stimulation provided by stimulation member 145 results in received response data from headgear 160 and/or biosignal monitor 165 that is different to response data received based on certain habituation stimulation being provided by stimulation member 145.

A change in response may comprise a change in the auditory-related regions of the subject's brain, which may be determined by measuring the changes in attenuation of the light received by detector optode 164 compared to the light emitted by source optode 162. A change in response may additionally or alternatively comprise a change in heart rate, heart rate variability, blood pressure, or breathing as measured by the source-detector pair of optodes 162 and 164 or as measured by the biosignal monitor 165. The effects of processor 120 executing processing module 156 are described below in further detail below with reference to FIG. 3.

Source optodes 162 may generate near-infrared (NIR) light, being light having a wavelength of between 650 and 1000 nm. In some embodiments, light may be generated at two or more different frequencies, with one frequency being absorbed more by the oxygenated haemoglobin (HbO) in the blood than by non-oxygenated haemoglobin (HbR), and one frequency being absorbed more by HbR than by HbO. In such embodiments, one frequency of light may be chosen such that the wavelength of the light is below 800 nm, and the other may be chosen to have a wavelength of above 800 nm. For example, according to some embodiments, one frequency may be around 760 nm, and the other frequency may be around 850 nm. In this document, these wavelengths will be referred to as the first wavelength and the second wavelength, respectively.

FIG. 2 shows a diagram 200 showing headgear 160 in position on a patient 201, where headgear 160 is an elastic cap with optodes positioned according to standard International EEG 10-10 system locations. Source optodes 162 (not shown) are arranged to be positioned in proximity to source positions 210 and detector optodes 164 (not shown) are arranged to be positioned in proximity to detector positions 220 when headgear 160 is worn correctly on a patient's head. While a single source optode 162 may illuminate a large area such that the reflected light may be measurable by multiple detector optodes 164, source optodes 162 and detector optodes 164 may be paired based on proximity. Measurement channels 250 highlight detector optode 164 and source optode 162 pairs which may be configured to cooperate such that the detector optode 164 collects reflected light from the paired source optode 162. In some embodiments, headgear 160 may comprise eight source optodes 162 and eight detector optodes 164.

According to some embodiments, optodes 162/164 may be arranged to be positioned over at least one of the bilateral superior temporal gyrus and the inferior frontal gyrus cortical areas of the patient's brain. According to some embodiments, optodes 162 may be arranged to be positioned over either of the left hemisphere 230, the right hemisphere 240, or both hemispheres 230/240. According to some embodiments, pairs of source optodes 164 and detector optodes 164 may be located around 0.5 to 5 cm apart.

Optodes 162/164 as arranged according to the source positions 210 and detector positions 220 illustrated in FIG. 2 allow for a number of different channels of data to be obtained. Each channel comprises a source optode 162 and a detector optode 164, although each source optode 162 and detector optode 164 may belong to more than one channel.

FIG. 3 shows a flow diagram 300 illustrating a method of assessing the hearing of a subject using system 100, as performed by processor 120 executing instructions stored in memory 150.

At step 310, processor 120 causes stimulus to be presented to a subject via stimulation member 145. According to some embodiments, this may involve processor 120 first retrieving an audio track from audio data 152, and communicating the retrieved audio track to sound output module 130, to cause sound output module 130 to communicate the audio track to sound generator 140. Sound generator 140 may then be caused to deliver the audio track to a subject via stimulation member 145. According to some embodiments, the audio track may have been generated by processor 120 executing stimulation generation module 153 to perform the method of FIG. 4, FIG. 5A and/or FIG. 5B. According to some embodiments, rather than retrieving a previously generated audio track, processor 120 may generate the audio track in real-time while delivering it to sound output module 130 to be presented to the subject via stimulation member 145, by performing the method of FIG. 4, FIG. 5A and/or FIG. 5B.

At step 320, processor 120 receives response data. According to some embodiments, processor 120 receives response data via data input module 180. According to some embodiments, the response data includes data generated by detector optodes 164. To receive the response data, processor 120 may first instruct light output module 170 to cause optodes 162 to emit light by sending source optodes 162 signals via transmission channels 168. Detector optodes 164 may be caused to generate signals in the form of light intensity readings based on an amount of light captured, and to output the signals to data input module 180 via measurement channels 166. In some embodiments, alternatively or additionally, the response data may include data received from biosignal monitor 165 via data input module 180. Where data is received from multiple sources, steps 320 to 347 may be performed for each set of data separately, and may be combined at step 350.

According to some embodiments, the response data may be categorised as either post-stimulus data or baseline data. According to some embodiments, the response data may be separated into samples of post-stimulus data and baseline data. Each sample of post-stimulus or baseline data may comprise a predetermined window or duration of data. Where data is received from multiple sources, response data categorised as post-stimulus data may be combined to form a combined single physiological response signal. Response data categorised as baseline data may be combined to form a combined single baseline response signal.

Post-stimulus data may include any response data received during delivery or within a predetermined duration directly before and/or after delivery of a discrimination stimulus signal to the subject. According to some embodiments, the duration may start 7, 5 or 3 seconds before the discrimination stimulus signal is delivered, and may end 10, 15, 25, or 30 seconds after the discrimination stimulus signal is delivered. For example, the duration may be from 5 seconds before to 27 seconds after stimulus delivery in some embodiments. In some embodiments, the duration may be between 5 seconds before to 20 seconds after stimulus delivery. In some embodiments, the duration may be between 3 seconds before to 15 seconds after stimulus delivery. In some embodiments, the duration may be between 3 seconds before to 11 seconds after. Samples of post-stimulus data may be obtained by extracting windows of data using the predetermined duration.

Baseline data may include fNIRS or other biosignal data received at any other time, not related to the stimulus onset. For example, baseline data may include response data received while no stimulus is being delivered to the subject, or while a stimulus signal unrelated to discrimination is being delivered to the subject. According to some embodiments, the baseline data may overlap with the post-stimulus data to some extent, but the baseline data may be selected not to exactly coincide with a post-stimulus data window. Samples of post-stimulus data may be obtained by extracting windows of data having a predetermined duration, where the window of data does not align with a window of post-stimulation data. According to some embodiments, the predetermined duration for the baseline data may be the same duration as that used for the post-stimulus data.

At step 330, processor 120 is caused to execute pre-processing module 154 to extract one or more desired sets of response data from the post-stimulus data received at step 320. According to some embodiments, processor 120 executing pre-processing module 154 may be caused to execute the method of FIG. 6, such that the extracted data includes hemodynamic response data. According to some embodiments, processor 120 executing pre-processing module 154 may be caused to execute the method of FIG. 7, such that the extracted data includes heart rate response data. According to some embodiments, processor 120 executing pre-processing module 154 may be caused to execute the method of both FIGS. 6 and 7, such that the extracted data includes both hemodynamic response data and heart rate response data. According to some embodiments, one or more other forms of response data may be extracted.

At step 335, processor 120 is caused to calculate a response quality of the data, which may be done by performing steps as described below with reference to FIGS. 6 and 7. In some embodiments, the response quality is calculated via the averaged standard error of mean of a collection of response epochs, as described below with reference to step 645 of FIG. 6. In some embodiments, the response quality is proportionate to the ratio of the power in a time segment of interest compared to the power in the whole signal. Once the response quality is calculated, processor 120 may further perform steps such as checking the data for bad channels, motion artefacts, statistically unlikely epochs, and bad regions, as described in further detail below with respect to steps 605, 615 and 640 of method 600 and steps 705 and 725 of method 700.

At step 340, processor 120 is caused to determine whether the data is of a sufficient quality based on the response quality calculated at step 335. If processor 120 determines that the data is not of sufficient quality, processor 120 may return to repeating step 310, presenting further stimulus so that further response data can be generated. If processor 120 determines that the data is of sufficient quality, processor 120 may proceed to step 345.

At step 345, processor 120 is caused to create a response model and/or fit the response features extracted at step 330 to a response model. According to some embodiments, this may comprise comparing the features extracted at step 330 with known discrimination response data, to determine whether the features correspond to a discrimination response. According to some embodiments, this may additionally or alternatively comprise comparing the features extracted at step 330 with known detection response data, to determine whether the features correspond to a detection response. For example, processor 120 may be caused to attempt to fit the extracted features to known response signals. According to some embodiments, processor 120 may be caused to model the response signal using an autoregressive integrative (ARI) model fit of the data as described in Barker et al. 2013 (Barker, J. W., Aarabi, A., & Huppert, T. J. (2013). Autoregressive model based algorithm for correcting motion and serially correlated errors in fNIRS. Biomed Opt Express, 4(8), 1366-1379. https://doi.org/10.1364/BOE.4.001366), or a real-time implementation of an adaptive general linear model as described in Abdelnour et al. 2009 (Abdelnour, A. F., & Huppert, T. (2009). Real-time imaging of human brain function by near-infrared spectroscopy using an adaptive general linear model. Neuroimage, 46(1), 133-143. https://doi.org/10.1016/j.neuroimage.2009.01.033). In some embodiments, processor 120 may be caused to model the response signal using a general linear model. According to some embodiments, processor 120 may be caused to process the extracted features using a computer learning method to determine whether the features match known discrimination response features and/or detection response features.

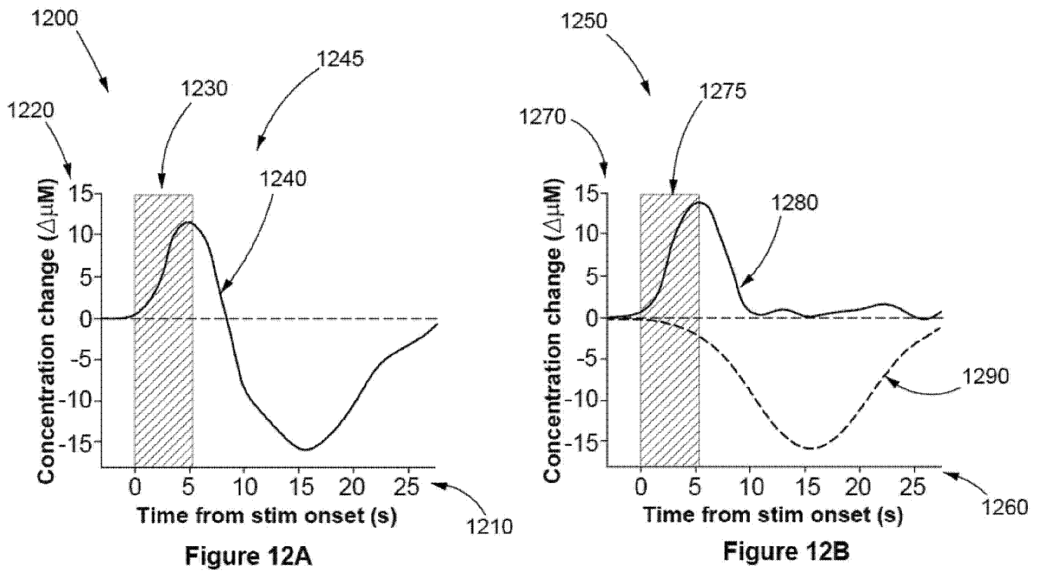

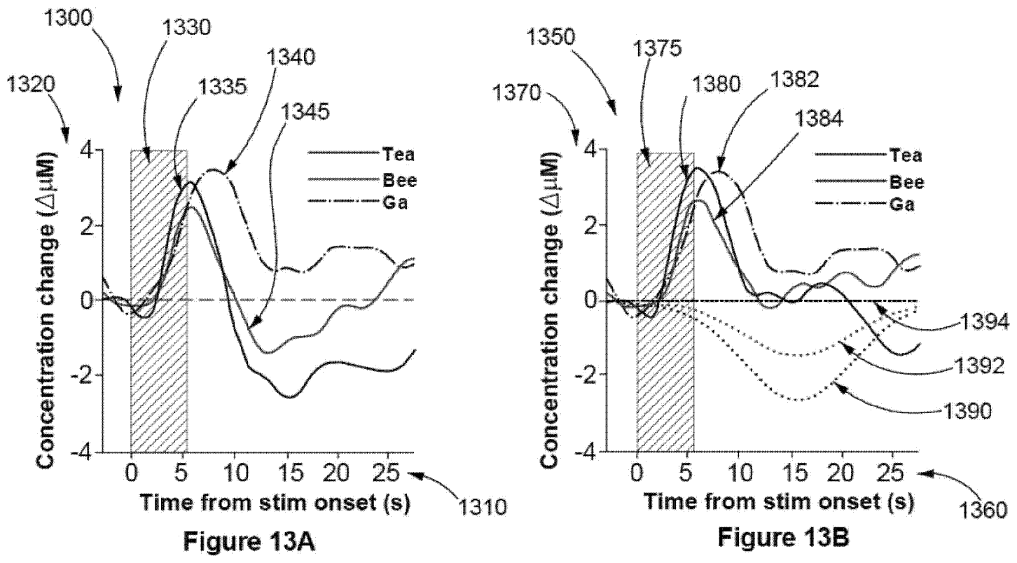

A stimulus may invoke more than one type of neural response in a patient. For example, an aural stimulus may activate the cortical auditory system of the patient while also activating a brain arousal response. These responses may occur simultaneously, or may overlap to some degree in some embodiments. In order to take into account these different types of neural responses, in some embodiments, processor 120 may be caused to model the neural response data as a sum of more than one concurrent neural response to the auditory stimulus. In some embodiments, the more than one neural response may include a response corresponding to the activation of the cortical auditory system by the auditory stimulus. In some embodiments, the more than one neural response may include a response corresponding to the activation of the brain arousal system by the stimulus. In some embodiments, the processor may be caused to separate the received response signal into separate signals, which can each be used to determine the discrimination and/or detection response, by fitting the response features of each separate signal to different response models. The separation may be, for example, achieved using an independent component analysis technique across different stimulus epochs. Examples showing a response separated into auditory and brain arousal responses is shown in FIGS. 12B and 13B, and described in further detail below. In some embodiments, the discrimination and/or detection response may be determined by comparing at least one feature of a separated response signal to the feature derived from the baseline or control data. In some embodiments, the features of the more than one separated neural response may be combined into a single feature for comparison to the baseline or control feature to determine the discrimination and/or detection response.

According to some embodiment, processor 120 may be caused to use a modelling technique that captures unique statistical properties in the neural response data pertaining to its neighbourhood covariance structure relative to aural stimulation onset, spanning the length of the expected response and across one or more responses. In some embodiments, the modelling technique summarises multiple known discrimination and/or detection responses in such a way that salient, contiguous overlapping data points in the response signals are captured whilst non-overlapping, non-correlated regions are suppressed. The statistical significance of this approximated response across multiple signals may be established by determining its dis-similarity to an approximation of arbitrary responses generated independent of the stimulus trigger.

In some embodiments, this may be done by using a stochastic process to model the response shape. A stochastic process may be used as a function approximation method that captures variation of several neural responses. The stochastic process encodes prior belief on how the responses can vary post stimuli as well as re-estimating the functional mean and covariance as new epochs are collected. The proposed multivariate form enables an analytically tractable solution when combining measurements from different regions exhibiting narrower variance across time where the responses overlap.

Constrained maximum likelihood optimization of the posterior function with respect to the hyperparameters encodes potential complexity of the signal processing task without handcrafting hyperparameters directly. In some embodiments, the stochastic process may be modelled as a Weiner, Gaussian, Bernoulli, Poisson, Gauss-Markov, Indian Buffet or a Chinese Restaurant process.

At step 347, processor 120 may be caused to extract statistical measures from the generated models. Statistical measures may include test statistics such as the t-values, chi-squared values or other model-specific test statistics.

Before deriving the statistical measures, processor 120 may first build a model using a stochastic process from the baseline response data as identified at step 320, where the model approximates a probabilistic function of what the baseline response looks like. The similarity of two processes may be established by running an equality test between the stochastic process built on the post-stimulus response as described at step 345, and the random process representing a control condition built based on the baseline response data. According to some embodiments, the baseline response data may comprise a randomly selected group of baseline response signals. According to some embodiments, the baseline response data may comprise data selected from periods where no stimulus was being delivered to the subject. According to some embodiments, the baseline response data may comprise data selected from periods where a detection response stimulus signal was being delivered to the subject. According to some embodiments, the baseline response data may comprise data selected from periods where no discrimination response stimulus signal was being delivered to the subject.

Processor 120 may subsequently use test statistics such as estimates of mean and covariance as well as statistics to evaluate whether the generated model for the post-stimulus data is different from the model generated from the control response data, by capturing differences in the two signal groups in a robust manner. To determine whether the subject exhibited a speech discrimination response, the post-stimulus response is measured after the presentation of a novel stimuli which in turn is preceded by the continual presentation of a habituation speech token, as outlined in further detail below with reference to FIGS. 4, 5A, 5B, 8 and 11. To determine whether the subject exhibited a speech detection response, the post-stimulus response is measured after the presentation of a stimuli which in turn is preceded by a period of silence, as outlined in further detail below with reference to FIGS. 4, 5A, and 11.

At step 350, processor 120 may optionally combine outcomes from multiple sources, if multiple sources exist. For example, processor 120 may one or more of an fNIRS response, biosignal response, EEG response, ABR (auditory brainstem response), CAEP (Cortical auditory evoked potentials) and ASSR (auditory steady-state response). The combination of multi-dimensional data may increase the accuracy and/or reliability of the measurements. Where a test statistic is calculated at step 347, processor 120 may use a statistical method to combine the test statistic for two sets of response features, for example, to allow for the results to be combined into a single determination. Response data categorised as post-stimulus data may be combined to form a combined single physiological response signal. Response data categorised as baseline data may be combined to form a combined single baseline response signal.

At step 352, processor 120 may be caused to test for whether a threshold degree of fit was achieved at step 345. A threshold degree of fit may indicate that the extracted features correspond to a discrimination and/or detection response. For example, processor 120 may use a matched filter or template matching strategy, by matching an expected response shape to the response, and using the resultant value in a statistical test. In some embodiments, the matched filter may take the shape of a Gaussian distribution, for example. In some embodiments, the response signals may be epoched, and the average of the odd and even epochs may be calculated by processor 120. A correlation between the two averaged values may be used to identify whether a discrimination and/or detection response was measured. In some embodiments, multiple statistical features extracted via a bootstrapping method during step 347 are combined to additionally generate a confidence measure as well as whether a significant response is present. For example, the statistical features may be permuted to create a collection of statistical measures. In some embodiments, the collection of statistical measures is used to generate a confidence level for the detected response or lack thereof.

At step 355, processor 120 determines whether a statistically significant response is present, based on the test performed at step 350. If processor 120 determines that no statistically significant response was present, processor 120 may return to repeating step 310, presenting further stimulus so that further response data can be generated. If processor 120 determines that a statistically significant response was present, processor 120 may proceed to step 360.

At step 360, processor 120 is caused to execute processing module 156 to determine an output. Specifically, processor 120 may be configured to use the extracted response data to determine whether the data corresponds to a discrimination response in a subject, indicating that the subject is able to discriminate between different sounds. In some embodiments, processor 120 may be alternatively or additionally configured to use the extracted response data to determine whether the data corresponds to a detection response in a subject, indicating that the subject is able to detect at least one sound. According to some embodiments, processor 120 may further be caused to output a result to user input module 112, or to communicate the result to an external computing system via communications module 190.

FIGS. 4, 5A and 5B illustrate two methods or paradigms for generating stimulus to be presented to a subject. According to some embodiments, the methods of FIGS. 4, 5A and/or 5B may form part of step 310 of FIG. 3.

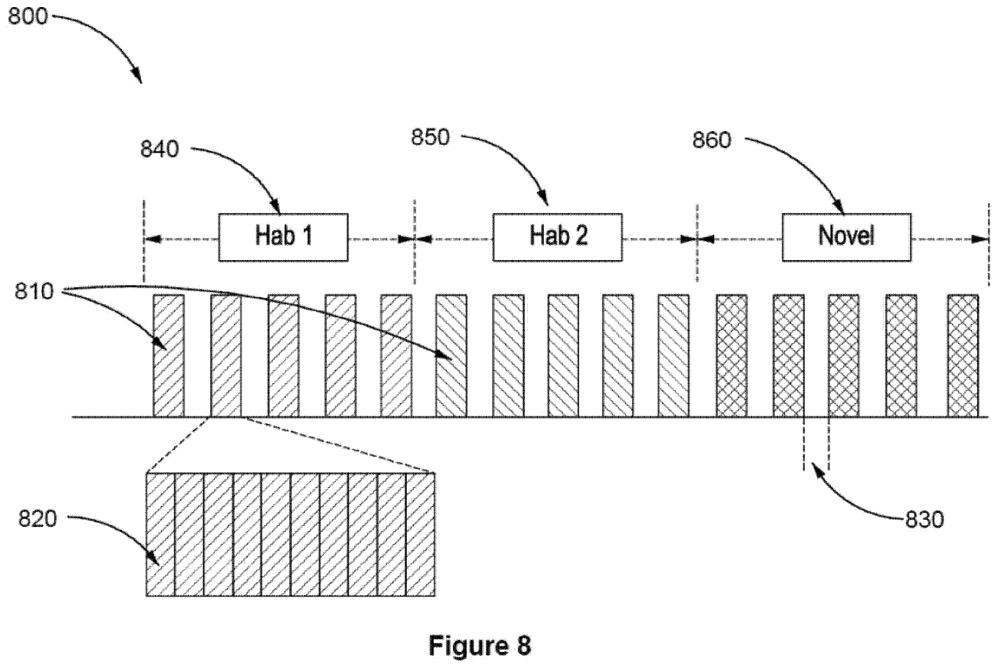

FIG. 4 shows a flow diagram 400 illustrating a first stimulus delivery method which may be used to generate stimulus to be delivered to a subject using system 100, as performed by processor 120 executing stimulus generation module 153. The first stimulus delivery method presents stimulation blocks interspersed with intervals of silence or having a silent baseline, as illustrated in FIG. 8. According to some embodiments, the method of FIG. 4 may be performed by assembling each of the audio stimuli into a single audio track and storing the track in audio data 152, to be retrieved and delivered by simulation device 145. In some embodiments, the method of FIG. 4 may be performed by retrieving audio stimuli from audio data 152 as separate audio tracks in real-time and communicating them to stimulation device 145 as they are required to be delivered to the subject.

At step 410, processor 120 is caused to retrieve at least one stored audio data file from audio data 152. According to some embodiments, a single audio file comprising a number of audio stimuli may be retrieved. In some embodiments, a plurality of audio files each comprising one or more audio stimuli may be retrieved.

According to some embodiments, the one or more audio tracks may comprise habituation and/or dishabituation audio signals. For example, the audio track may comprise a segment of audio data having a first sound, followed by a segment of audio data having a second sound. The first sound may be a habituation audio signal, and the second sound may be a dishabituation audio signal. According to some embodiments, a first audio track may comprise habituation audio signals, and a second audio track may comprise dishabituation audio signals. According to some embodiments, the audio track may comprise multiple segments of habituation audio signals. According to some embodiments, the audio track may comprise multiple segments of dishabituation audio signals. According to some embodiments, the segments may be separated by periods of silence.

At step 420, processor 120 causes a habituation stimulus to be delivered to a subject via stimulation member 145. Processor 120 may do this by communicating a retrieved audio track to sound output module 130, which is caused to communicate the audio track to sound generator 140. Sound generator 140 causes the audio track to play via stimulation member 145.

According to some embodiments, the delivered stimuli may be a block of stimuli comprising a repeated voiced syllable or word. For example, the stimuli may comprise a human voice repeating a word or syllable such as “ba”, “tea”, “she”, “see”, “boo”, “bee”, “ga”, “pa” or “ma”, in some embodiments. According to some embodiments, the block of stimuli may be at least 0.5 seconds long, and may be around 5 seconds in some embodiments. For example, the block of stimuli may be around 5.4 seconds long. The audio may be delivered at any intensity that is audible to the subject without being uncomfortably loud. For example, the stimulus intensity may be around 65 dB SPL in some embodiments.

At step 430, processor 120 causes a silence interval to be delivered to the subject via the stimulation member 145. A silence interval may comprise no sound or audio being delivered by stimulation member 145. According to some embodiments, this may occur without further intervention by processor 120, by sound generator 140 continuing to cause the audio track to play via stimulation member 145, where the audio track comprises both habituation stimulus and silence intervals. In some embodiments, this may occur by processor 120 pausing or stopping any audio track from being communicated to sound generator 140 or being delivered by stimulation member 145.

According to some embodiments, the silence interval may be at least 5 seconds long. In some embodiments, the silence interval may be around 9 seconds, for example.

According to some embodiments, the silence interval may be selected to be long enough to allow for any response from presentation of the habituation stimuli at step 420 to have returned to baseline before a new stimulus is presented. In order to increase the effect of the habituation stimulus pattern, the variation in duration of silence intervals presented to the subject may be zero, so that each silence interval presented to the subject is of the same duration.

At step 440, processor 120 determines whether further habituation stimulus is required to be delivered. In some embodiments, this may be done by checking whether further habituation stimulus data exists in audio data 152 which has not yet been delivered. In some embodiments, this may be by ascertaining whether a predetermined number of habituation stimulus sounds have been delivered to the subject. For example, according to some embodiments, between 3 and 20 blocks of habituation stimulus may be presented to the subject. In some embodiments, around 10 blocks of habituation stimulus may be presented to the subject. The number of stimulus blocks may be chosen such that the subject becomes habituated to the stimulus. Processor 120 may use a counter to track the number of stimulus blocks delivered to the subject, and may compare the value of the counter to a predetermined value to determine whether further stimulus blocks should be delivered, for example.

If processor 120 determines that further habituation stimulus is to be delivered, processor 120 proceeds to repeat method 400 from step 420. If no further habituation stimulus is to be delivered, processor 120 proceeds to execute method 400 from step 450. According to some embodiments, a predetermined number of habituation stimulus blocks may be recorded in an audio track, such that processor 120 isn't required to make the determination at step 440.

At step 450, processor 120 causes dishabituation stimuli to be delivered to a subject via stimulation member 145. According to some embodiments, this may occur without further intervention by processor 120, by sound generator 140 continuing to cause the audio track to play via stimulation member 145, where the audio track comprises both habituation and dishabituation audio signals. In some alternative embodiments, processor 120 may cause dishabituation stimuli to be delivered by communicating a second retrieved audio track having the dishabituation audio signals to sound output module 130, which is caused to communicate the audio track to sound generator 140. Sound generator 140 causes the audio track to play via stimulation member 145.

According to some embodiments, the delivered stimuli may be a block of stimuli comprising a repeated voiced syllable or word. For example, the stimuli may comprise a human voice repeating a syllable or word such as “ba”, “tea”, “she”, “see”, “boo”, “bee”, “ga”, “pa” or “ma”, in some embodiments. According to some embodiments, the dishabituation stimulus may be selected to contrast with the habituation stimulus. For example, the dishabituation stimulus may be selected to differ from the habituation stimulus in an acoustic feature such as one or more of spectral features, temporal features, place or manner of articulation, voicing, frication, or tone. According to some embodiments, habituation and dishabituation stimuli may be stored together in contrasting pairs. According to some embodiments, the block of stimuli may comprise both the habituation and dishabituation stimuli, which may be presented in an alternating pattern. Some examples of contrasting syllable or word pairs that may be used as habituation and dishabituation stimuli may include ba/tea, she/see, boo/bee, ba/bee, ba/ga, ba/pa, and ba/ma sounds.

According to some embodiments, the block of stimuli may be at least 0.5 seconds long, and may be around 5 seconds in some embodiments. For example, the block of stimuli may be around 5.4 seconds long. The audio may be delivered at any intensity that is audible to the subject without being uncomfortably loud. For example, the stimulus intensity may be around 65 dB SPL in some embodiments.

At step 460, processor 120 causes a silence interval to be delivered to the subject via the stimulation member 145. A silence interval may comprise no sound or audio being delivered by stimulation member 145. According to some embodiments, this may occur without further intervention by processor 120, by sound generator 140 continuing to cause the audio track to play via stimulation member 145, where the audio track comprises both dishabituation stimulus and silence intervals. In some embodiments, this may occur by processor 120 pausing or stopping any audio track from being communicated to sound generator 140 or being delivered by stimulation member 145.

According to some embodiments, the silence interval may be at least 5 seconds long. In some embodiments, the silence interval may be around 9 seconds, for example. According to some embodiments, the silence interval may be selected to be long enough to allow for any response from presentation of the dishabituation stimuli at step 450 to have returned to baseline before a new stimulus is presented.

At step 470, processor 120 determines whether further dishabituation stimulus is required to be delivered. In some embodiments, this may be done by checking whether further dishabituation stimulus data exists in audio data 152 which has not yet been delivered. In some embodiments, this may be by ascertaining whether a predetermined number of dishabituation stimulus sounds have been delivered to the subject. For example, according to some embodiments, between 1 and 10 blocks of dishabituation stimulus may be presented to the subject. In some embodiments, around 5 blocks of dishabituation stimulus may be presented to the subject. Processor 120 may use a counter to track the number of stimulus blocks delivered to the subject, and may compare the value of the counter to a predetermined value to determine whether further stimulus blocks should be delivered, for example.

If processor 120 determines that further dishabituation stimulus is to be delivered, processor 120 proceeds to repeat method 400 from step 450. If no further dishabituation stimulus is to be delivered, processor 120 proceeds to execute method 400 from step 320. According to some embodiments, a predetermined number of dishabituation stimulus blocks may be recorded in an audio track, such that processor 120 isn't required to make the determination at step 470.

At step 320, processor 120 is caused to receive response data, as described above with reference method 300. Processor 120 may proceed to process the received data according to method 300, to determine whether the stimulus delivered by performance of method 400 causes a discrimination response.

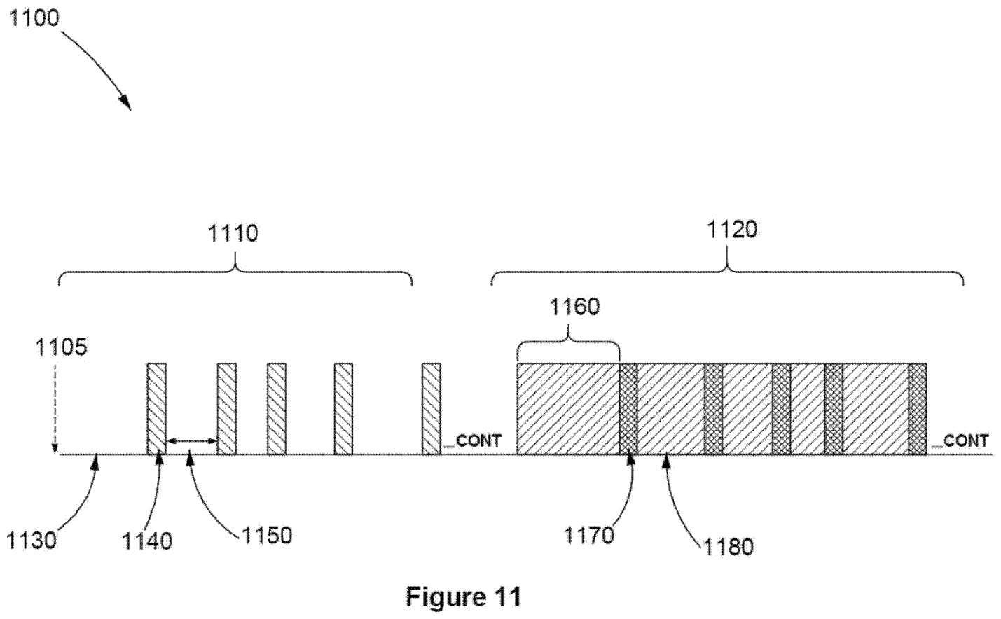

FIGS. 5A and 5B shows flow diagrams 500 and 550 illustrating a second stimulus delivery method which may be used to generate stimulus to be delivered to a subject using system 100, as performed by processor 120 executing stimulus generation module 153. The second stimulus method comprises a habituation portion, illustrated in FIG. 5A, which presents stimulation blocks interspersed with silence intervals; and a dishabituation portion, illustrated in FIG. 5B, which presents stimulation blocks interspersed with non-silent intervals; as illustrated in FIG. 11.

According to some embodiments, methods 500 and/or 550 may be performed as part of step 310 of method 300. Method 500 may be performed to determine whether a subject exhibits a detection response, while method 550 may be performed to determine whether a subject exhibits a discrimination response.

Turning to FIG. 5A, at step 510, processor 120 is caused to retrieve at least one stored audio data file from audio data 152. According to some embodiments, a single audio file comprising a number of audio stimuli may be retrieved. In some embodiments, a plurality of audio files each comprising one or more audio stimuli may be retrieved.

According to some embodiments, the one or more audio tracks may comprise one or more detection, habituation and/or dishabituation audio signals. Detection audio signals may be delivered to a subject interspersed with silent intervals to establish a detection response in the subject. Habituation and dishabituation signals may subsequently be delivered to the subject without a silence interval to establish a discrimination response.

According to some embodiments, the audio track may comprise a segment of audio data having a first sound, followed by a segment of audio data having a second sound, followed by a segment of audio data having a third sound. The first sound may be a detection audio signal, the second sound may be a habituation audio signal, and the third sound may be a dishabituation audio signal. According to some embodiments, a first audio track may comprise detection audio signals, a second audio track may comprise habituation audio signals, and a third audio track may comprise dishabituation audio signals. According to some embodiments, the audio track may comprise multiple segments of detection, habituation and/or dishabituation audio signals. According to some embodiments, some segments may be separated by periods of silence.

At step 520, processor 120 causes a detection stimulus to be delivered to a subject via stimulation member 145. Processor 120 may do this by communicating a retrieved audio track to sound output module 130, which is caused to communicate the audio track to sound generator 140. Sound generator 140 causes the audio track to play via stimulation member 145.

According to some embodiments, the delivered stimuli may be a block of stimuli comprising a repeated voiced syllable or word. For example, the stimuli may comprise a human voice repeating a syllable or word such as “ba”, “tea”, “she”, “see”, “boo”, “bee”, “ga”, “pa” or “ma”, in some embodiments. According to some embodiments, the block of stimuli may be at least 0.5 seconds long, and may be around 5 seconds in some embodiments. For example, the block of stimuli may be around 5.4 seconds long. The audio may be delivered at any intensity that is audible to the subject without being uncomfortably loud. For example, the stimulus intensity may be around 65 dB SPL in some embodiments.

At step 530, processor 120 causes a silence interval to be delivered to the subject via the stimulation member 145. A silence interval may comprise no sound or audio being delivered by stimulation member 145. According to some embodiments, this may occur without further intervention by processor 120, by sound generator 140 continuing to cause the audio track to play via stimulation member 145, where the audio track comprises both habituation stimulus and silence intervals. In some embodiments, this may occur by processor 120 pausing or stopping any audio track from being communicated to sound generator 140 or being delivered by stimulation member 145.

According to some embodiments, the silence interval may be at least 5 seconds long. In some embodiments, the silence interval may be between 20 and 35 seconds long, for example. According to some embodiments, the silence interval may be selected to be long enough to allow for any response from presentation of the detection stimuli at step 520 to have returned to baseline before a new stimulus is presented.

At step 540, processor 120 determines whether further detection stimulus is required to be delivered. In some embodiments, this may be done by checking whether further detection stimulus data exists in audio data 152 which has not yet been delivered. In some embodiments, this may be by ascertaining whether a predetermined number of detection stimulus sounds have been delivered to the subject. For example, according to some embodiments, between 1 and 10 blocks of detection stimulus may be presented to the subject. In some embodiments, around 5 blocks of detection stimulus may be presented to the subject. The number of detection blocks may be chosen such that a consistent detection response can be measured in the subject. Processor 120 may use a counter to track the number of stimulus blocks delivered to the subject, and may compared the value of the counter to a predetermined value to determine whether further stimulus blocks should be delivered, for example.

If processor 120 determines that further detection stimulus is to be delivered, processor 120 proceeds to repeat method 500 from step 520. If no further detection stimulus is to be delivered, processor 120 proceeds to execute method 500 from step 320. According to some embodiments, a predetermined number of detection stimulus blocks may be recorded in an audio track, such that processor 120 isn't required to make the determination at step 540.

At step 320, processor 120 is caused to receive response data, as described above with reference method 300. Processor 120 may proceed to process the received data according to method 300, to determine whether the stimulus delivered by performance of method 500 caused a detection response.

Where a detection response was satisfactorily established, processor 120 may subsequently be caused to repeat method 300 to establish a discrimination response. In this case, processor 120 may be caused to perform method 550 during step 310 of the second iteration of method 300. Method 550 is described below with reference to FIG. 5B.

Turning to FIG. 5B, at step 510, processor 120 is caused to retrieve at least one stored audio data file from audio data 152. According to some embodiments, a single audio file comprising a number of audio stimuli may be retrieved. In some embodiments, a plurality of audio files each comprising one or more audio stimuli may be retrieved. In some embodiments, the audio file may have previously been retrieved during step 510 of method 500.

According to some embodiments, the one or more audio tracks may comprise one or more detection, habituation and/or dishabituation audio signals. Detection audio signals may be delivered to a subject interspersed with silent intervals to establish a detection response in the subject. Habituation and dishabituation signals may subsequently be delivered to the subject without a silence interval to establish a discrimination response.

According to some embodiments, the audio track may comprise a segment of audio data having a first sound, followed by a segment of audio data having a second sound, followed by a segment of audio data having a third sound. The first sound may be a detection audio signal, the second sound may be a habituation audio signal, and the third sound may be a dishabituation audio signal. According to some embodiments, a first audio track may comprise detection audio signals, a second audio track may comprise habituation audio signals, and a third audio track may comprise dishabituation audio signals. According to some embodiments, the audio track may comprise multiple segments of detection, habituation and/or dishabituation audio signals. According to some embodiments, some segments may be separated by periods of silence.

At step 560, processor 120 causes a habituation stimulus to be delivered to a subject via stimulation member 145. Processor 120 may do this by communicating a retrieved audio track to sound output module 130, which is caused to communicate the audio track to sound generator 140. Sound generator 140 causes the audio track to play via stimulation member 145.

According to some embodiments, the delivered stimuli may be a block of stimuli comprising a repeated voiced syllable or word. For example, the stimuli may comprise a human voice repeating a syllable or word such as “ba”, “tea”, “she”, “see”, “boo”, “bee”, “ga”, “pa” or “ma”, in some embodiments. The voiced syllable may be the same as that used for the detection stimulus in some embodiments. In some embodiments, the stimulus may be different to that used for the detection stimulus. According to some embodiments, the block of stimuli may be at least 5 seconds long, and may be between and 35 seconds in some embodiments. The audio may be delivered at any intensity that is audible to the subject without being uncomfortably loud. For example, the stimulus intensity may be around 65 dB SPL in some embodiments.

At step 570, processor 120 causes dishabituation stimuli to be delivered to a subject via stimulation member 145. According to some embodiments, this may occur without further intervention by processor 120, by sound generator 140 continuing to cause the audio track to play via stimulation member 145, where the audio track comprises both habituation and dishabituation audio signals. In some alternative embodiments, processor 120 may cause dishabituation stimuli to be delivered by communicating a second retrieved audio track having the dishabituation audio signals to sound output module 130, which is caused to communicate the audio track to sound generator 140. Sound generator 140 causes the audio track to play via stimulation member 145.

According to some embodiments, the delivered stimuli may be a block of stimuli comprising one or more repeated voiced syllables or words. For example, the stimuli may comprise a human voice repeating a syllable or word such as “ba”, “tea”, “she”, “see”, “boo”, “bee”, “ga”, “pa” or “ma”, in some embodiments. According to some embodiments, the stimuli may comprise a human voice repeating a combination of syllables or words, such as “Tea/Ba”, “Bee/Ba”, “Ga/Ba”, “Pa/Ba” or “Ma/Ba” in some embodiments. According to some embodiments, the stimuli may comprise a human voice repeating a combination of speech stimuli where at least one of the stimuli is the habituation stimuli, and the other stimuli is a contrasting stimuli.

According to some embodiments, at least one syllable, word or sound of the dishabituation stimulus may be selected to contrast with the habituation stimulus. For example, the syllable, word or sound may be selected to differ from the habituation stimulus in an acoustic feature such as one or more of spectral features, temporal features, place or manner of articulation, voicing, frication, or tone. According to some embodiments, habituation and dishabituation stimuli may be stored together in contrasting pairs.

According to some embodiments, the block of stimuli may be at least 0.5 seconds long, and may be around 5 seconds in some embodiments. For example, the block of stimuli may be around 5.4 seconds long. The audio may be delivered at any intensity that is audible to the subject without being uncomfortably loud. For example, the stimulus intensity may be around 65 dB SPL in some embodiments.

At step 580, processor 120 determines whether further stimulus is required to be delivered. In some embodiments, this may be done by checking whether further stimulus data exists in audio data 152 which has not yet been delivered. In some embodiments, this may be by ascertaining whether a predetermined number of stimulus sounds have been delivered to the subject. For example, according to some embodiments, between 1 and 10 blocks of dishabituation stimulus may be presented to the subject. In some embodiments, around 5 blocks of dishabituation stimulus may be presented to the subject. Processor 120 may use a counter to track the number of stimulus blocks delivered to the subject, and may compared the value of the counter to a predetermined value to determine whether further stimulus blocks should be delivered, for example.

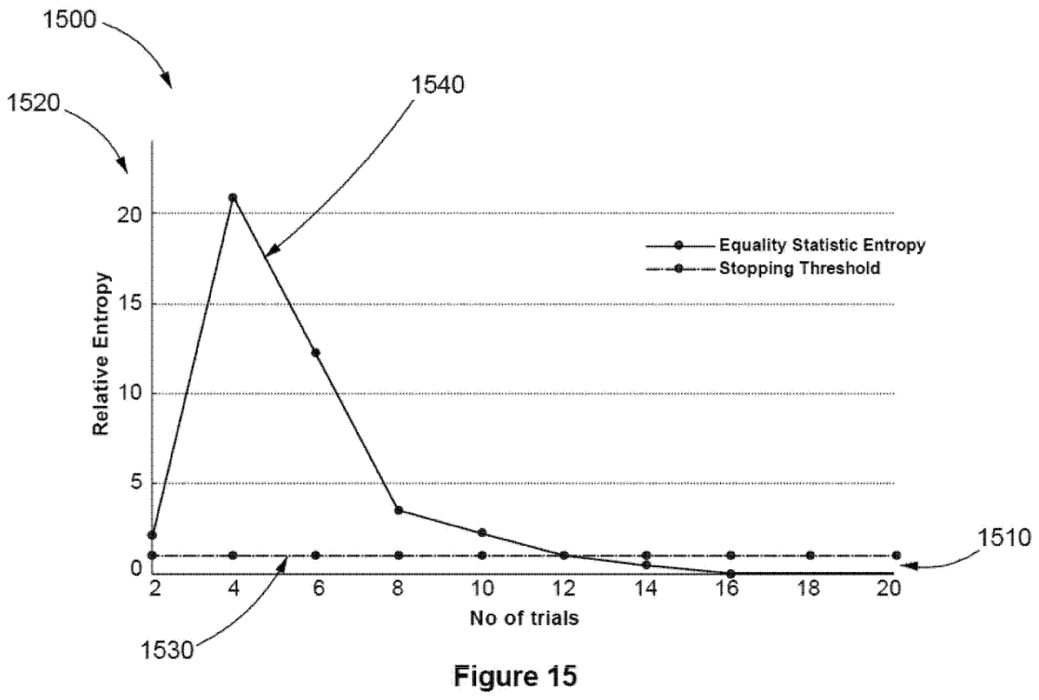

In another embodiment, at step 580 processor 120 calculates a stopping criterion based on the data that has already been acquired, and determines that further stimulus is required only if the stopping criterion has not been met. For example, the stopping criterion may be determined based on the statistical measures generated at step 347 of method 300. An example graph illustrating the determination of a stopping criterion is shown in FIG. 15, and described in further detail below. When processor 120 is caused, at step 347 of method 300, to generate a distribution of statistical measures from the current response data, processor 120 may be caused to characterize the difference in this distribution and the distribution generated from the previous data loop of steps 560 to 580. If processor 120 determines that the difference between the distributions, characterized as relative entropy between the distributions 1540, is less than a predetermined threshold 1530, then processor 120 determines that the stopping criterion is reached, and no further stimuli should be presented.

If processor 120 determines that further stimulus is to be delivered, processor 120 proceeds to repeat method 550 from step 560. If no further habituation stimulus is to be delivered, processor 120 proceeds to execute method 550 from step 320. According to some embodiments, a predetermined number of stimulus blocks may be recorded in an audio track, such that processor 120 isn't required to make the determination at step 580.

At step 320, processor 120 is caused to receive response data, as described above with reference to method 300. Processor 120 may proceed to process the received data according to method 300, to determine whether the stimulus delivered by performance of method 550 caused a discrimination response.

FIGS. 6 and 7 relate to methods of pre-processing data received after performing methods 400, 500 or 550, and receiving data generated based on the subject's response to the provided stimulation. The methods of FIGS. 6 and 7 may form part of step 330 of method 300.

FIG. 6 shows a flow diagram 600 illustrating a method of extracting hemodynamic response features, as performed by processor 120 executing pre-processing module 154. While the pre-processing steps in diagram 600 are illustrated and described as being performed in a particular order, it is to be understood that in some embodiments, the order of the steps may be varied. Furthermore, in some embodiments only a selection of the pre-processing steps might be performed.

Method 600 begins with processor 120 receiving response data from a subject at step 320, as described above with reference to method 300 of FIG. 3. Specifically, step 320 of method 600 requires that the received response data is hemodynamic response data, or response data generated by detector optodes 164. To receive the response data, processor 120 may first instruct light output module 170 to cause optodes 162 to emit light by sending source optodes 162 signals via transmission channels 168. Detector optodes 164 may be caused to generate signals in the form of light intensity readings based on an amount of light captured, and to output the signals to data input module 180 via measurement channels 166.

Processor 120 executing pre-processing module 154 is then caused to process the data by removing noise and unwanted signal elements from the response data. According to some embodiments, these may include signal elements such as those caused by breathing of the patient, the heartbeat of the patient, a Mayer wave, a motion artefact, non-hearing related brain activity, and the data collection apparatus, such as measurement noise generated by the hardware. In some embodiments, the signal elements caused by breathing or heartbeats may be kept for further analysis, as described below with respect to FIG. 7.

To do this, at step 605 processor 120 executing pre-processing module 154 is caused to exclude channels of data from further analysis if they are considered to be bad channels. According to some embodiments, bad channels may be the result of channels in which the scalp of the patient and the optodes 162/164 are not well coupled. The identification and removal of bad channels may be done in a number of ways.

According to some embodiments, channels with high gains may be considered bad channels and excluded from analysis, as high gains may correspond to low light intensity received by detector optodes 164. For example, if the connection between a detector optode 164 and the scalp of a patient is blocked by hair, or if the optode 164 is otherwise not in good contact with the skin, then the light received by detector optode 164 will have a relatively low intensity. Device 110 may be configured to automatically increase the gain for detector 164 where the signal being generated by detector 164 is low in magnitude. If this gain value is too high, this may indicate that there is poor coupling between detector 164 and the scalp, and that the data from that detector 164 should be discarded. Based on this, according to some embodiments, step 605 may include discarding channel values where the gain for the channel is above a predetermined threshold value. Similarly if the automatically-set gain is very low, it may indicate that the source optode 162 may not be correctly placed against the scalp, and needs to be repositioned or the channel discarded. According to some embodiments, channels with gains over 7 (as indicated by the NIRx NIRScout system) may be discarded, as this may indicate inadequate scalp-electrode connection. According to some embodiments, channels with a gain under a predetermined threshold, or equal to a predetermined threshold, may also be discarded. For example, according to some embodiments, channels with a gain of 0 may be discarded.

According to some embodiments, channels with low correlation between the first wavelength and the second wavelength may also be considered bad channels and discarded, as described in Pollonini, L., Olds, C., Abaya, H., Bortfeld, H., Beauchamp, M. S., & Oghalai, J. S. (2014), “Auditory cortex activation to natural speech and simulated cochlear implant speech measured with functional near-infrared spectroscopy”, Hearing research, 309, 84-93. Low correlation between the first wavelength and the second wavelength may be another indication of poor coupling between a detector 164 and the scalp of the patient. To calculate the correlation, in some embodiments, data may first be filtered using a narrow bandpass filter, which may be used to filter out all signals apart from those in the heartbeat range, which may be signals between 0.5-1.5 Hz, or between 0.5 Hz and 2.5 Hz, for example. The remaining signal is dominated by the heartbeat signal, and is commonly the strongest signal in the raw fNIRS data received from detectors 164, and therefore should show up strongly in the signals for both the first wavelength and the second wavelength if both source 162 and detector 164 are well-coupled with the skin of the patient.

If the first wavelength and the second wavelength are strongly correlated, this indicates that the coupling between the scalp and detector 164 is sufficiently strong.

According to some embodiments, the correlation between the first and the second wavelength may be determined to be the scalp coupling index (SCI). The SCI may be calculated as the correlation between the two detected signals at the first wavelength and at the second wavelength, and filtered to a range that would mainly include heart beat data, as described above. For example, the SCI may be calculated as the correlation between the two detected signals at 760 and 850 nm and band-pass filtered between 0.5 and 2.5 Hz, in some embodiments. According to some embodiments, channels with SCIs lower than a predetermined threshold may be rejected. For example, according to some embodiments, channels with an SCI of less than 0.8 may be rejected. According to some embodiments, channels with an SCI of less than 0.7 may be rejected. According to some embodiments, channels with an SCI of less than 0.6 may be rejected. According to some embodiments, channels with an SCI of less than 0.5 may be rejected.

At step 610, processor 120 executing pre-processing module 154 is caused to convert the first wavelength raw data and the second wavelength raw data of the remaining channels into a unit-less measure of changes in optical density over time. This step may be performed as described in Huppert, T. J., Diamond, S. G., Franceschini, M. A., & Boas, D. A. (2009), “HomER: a review of time-series analysis methods for near-infrared spectroscopy of the brain”, Appl Opt, 48(10), D280-298.

At step 615, processor 120 executing pre-processing module 154 is caused to remove motion artefacts in the optical density data. According to some embodiments, this may be done by performing temporal derivative distribution repair (TDDR), as described in Fishburn F. A., Ludlum R. S., Vaidya C. J., Medvedev A. V. (2019) “Temporal Derivative Distribution Repair (TDDR): A motion correction method for fNIRS”, Neuroimage. 2019 1; 184:171-179. According to some embodiments, motion artefacts may manifest as spike-shaped artefacts in the data. Motion artefacts may be removed using wavelets, as described in Molavi, B., & Dumont, G. A. (2012), “Wavelet-based motion artefact removal for functional near-infrared spectroscopy”, Physiological measurement, 33(2), 259. In some embodiments, motion artefacts may be removed using threshold-crossing detection and spline-interpolation.

According to some embodiments, motion artefacts may also or alternatively be removed using techniques such as outlier detection using analysis of studentised residuals, use of principal component analysis (PCA) to remove signals with high covariance across multiple source-detector pairs and across optical wavelengths, Wiener filtering and autoregression models, as described in Huppert, T. J., Diamond, S. G., Franceschini, M. A., & Boas, D. A. (2009), “HomER: a review of time-series analysis methods for near-infrared spectroscopy of the brain”, Appl Opt, 48(10), D280-298.

At step 620, processor 120 executing pre-processing module 154 is caused to pass the signals generated at step 615 through a bandpass filter to remove drift, broadband noise and/or systemic physiological responses such as heartbeat, respiration rhythm, and systemic blood pressure changes. According to some embodiments, the bandpass filter may be a Butterworth filter. In some embodiments, the bandpass filter may be any finite impulse response (FIR) or infinite impulse response (IIR) filter. According to some embodiments, the bandpass filter may be a 0.01 to 1 Hz bandpass filter. According to some embodiments, step 620 may also or alternatively involve the removal of physiological signals in other ways, such as using other filtering methods, adaptive filtering or remote measurement of the signals to subtract them, as described in Kamran, M. A., Mannan, M. M. N., & Jeong, M. Y. (2016), “Cortical Signal Analysis and Advances in Functional Near-Infrared Spectroscopy Signal: A Review”, Front Hum Neurosci, 10, and Huppert, T. J., Diamond, S. G., Franceschini, M. A., & Boas, D. A. (2009) “HomER: a review of time-series analysis methods for near-infrared spectroscopy of the brain”, Appl Opt, 48(10), D280-298.

At step 625, processor 120 executing pre-processing module 154 is caused to convert the signals generated at step 620 to HbO and HbR concentration change signals, using the modified Beer-Lambert law as described in Delpy, D. T., Cope, M., van der Zee, P., Arridge, S., Wray, S., & Wyatt, J. (1988), “Estimation of optical pathlength through tissue from direct time of flight measurement”, Physics in medicine and biology, 3 (12), 1433. Step 625 may involve converting the optical density data as derived from the signals received from optodes 164 to concentration change units, taking into account the channel length, being the distance between the source 162 and the detector 164 optodes.

At optional step 630, in order to remove the contribution of skin and scalp signals from the long channels, processor 120 executing pre-processing module 154 may be caused to remove short channel data from the long channel data, either directly by subtraction or by using a general linear model (GLM). In general, the shorter the distance between an optode pair 162/164, the shallower the area from which the signal is recorded. Therefore, very short channels measure activity only from the blood vessels in the skin and scalp. Very short channels may comprise source and detector pairs positioned around 1.5 cm or less apart. The skin and scalp signals may include signals relating to heartbeat, breathing and blood pressure.

According to some embodiments, principle component analysis (PCA) may be carried out across the short channels only. The first principle component (PC) across the short channels may represent activity common to all the short channels, which can then be included as a term in the general linear model of the long channel data and then effectively removed. According to some embodiments, this step may be carried out based on the methods outlined in Sato, T., Nambu, I., Takeda, K., Aihara, T., Yamashita, O., Isogaya, Y., Osu, R. (2016), “Reduction of global interference of scalp-hemodynamics in functional near-infrared spectroscopy using short distance probes”, NeuroImage, 141, 120-132.

Additionally or alternatively at step 630, information from additional sensors such as pulse oximeters, for example, can be used to remove the residual systemic noise, in some embodiments. The data from such additional sensors can be included as a regressor in the general linear model and then effectively removed. According to some embodiments, this step may be carried out based on the methods outlines in Sutoko, S., Chan, Y. L., Obata, A., Sato, H., Maki, A., Numata, T., Funane, T., Atsumori, H., Kiguchi, M., Tang, T. B., Li, Y., Frederick, B. D., Tong, Y. (2019), “Denoising of neuronal signal from mixed systemic low-frequency oscillation using peripheral measurement as noise regressor in near-infrared imaging.”, Neurophotonics 6, 015001.

At optional step 635, processor 120 executing pre-processing module 154 may be caused to epoch the time series of HbO and HbR concentration change data determined at step 630. Each epoch may be from around-5 to 30 seconds relative to the onset time of the stimulus. According to some embodiments, the epochs may be selected to avoid extending into the next stimulus period. According to some embodiments, other epoch time values may be used depending on stimulus length and inter-stimulus interval length. According to some embodiments, step 635 may further comprise performing baseline correction and/or detrending.

At optional step 640, processor 120 executing pre-processing module 154 may be caused to exclude epochs with statistically unlikely concentration change values. For example, a set of epochs may be rejected if their values are outside of 3 standard deviations from the mean of the epoch time-domain average. According to some specific embodiments, epochs with early stimulation phase values within the range of mean plus 2.5 standard deviations (across trials) may be included, and all other epochs may be excluded. The early stimulation phase may be defined as from −5 to +2 seconds in some embodiments. According to some embodiments, step 640 may be performed as described in Huppert, T. J., Diamond, S. G., Franceschini, M. A., & Boas, D. A. (2009), “HomER: a review of time-series analysis methods for near-infrared spectroscopy of the brain”, Appl Opt, 48(10), D280-298. The excluded epochs may relate to movement artefacts and noise.

According to some embodiments, at optional step 640 processor 120 may further be configured to exclude epochs with a peak spectral power outside a particular threshold, as described in Pollonini, L., Bortfeld, H., & Oghalai, J. S. (2016). PHOEBE: a method for real time mapping of optodes-scalp coupling in functional near-infrared spectroscopy. Biomed Opt Express, 7(12), 5104-5119. https://doi.org/10.1364/boe.7.005104. For example, according to some embodiments, epochs with a peak spectral power of more than 0.15 may be excluded.

At optional step 645, where multiple different stimuli have been presented during the measurements, processor 120 executing pre-processing module 154 may be caused to separately average data resulting from each of the stimulations of the same type.

At optional step 650, where overlapping channels of optodes 162/164 were used, processor 120 executing pre-processing module 154 may be caused to average the averaged responses from the overlapping channels. Averaging data across overlapping channels may reduce noise in the data.

At optional step 655, processor 120 executing pre-processing module 154 may be caused to construct regions of interest (ROIs) based on the positions of the optodes 162/164. According to some embodiments, two or more neighbouring channels may be combined into one ROI. Channels to group as ROIs may be selected according to similar response waveform patterns, for example. Channels to group as ROIs may be also be selected according to pre-determined anatomical or functional considerations

At step 660, processor 120 executing pre-processing module 154 is caused to automatically extract measures from the response signals. These measures may include a calculated magnitude of the peak of the signal, if the response shows single peak, or a calculated mean magnitude in an early and/or late window of the signal. According to some embodiments, an early window may be a window of around 3 to 9 seconds from the stimulation onset time, and a late window may be a window of around 14 to 20 seconds from the stimulation onset time. According to some embodiments, the response magnitude may be averaged over multiple time windows of various durations and centre times covering all or part of the epoched time window. According to some embodiments, an early window may be a window of around 0 to 6 seconds from the stimulation onset time, and a late window may be a window of around 24 to 30 seconds from the stimulation onset time. According to some embodiments, the measures may also or alternatively include a calculated time to the peak of the signal, and/or a width of the peak of the signal.

The extracted measures may be used by processor 120 in executing step 340 of method 300 to determine an output, as described in further detail above with reference to FIG. 3.

FIG. 7 shows a flow diagram 700 illustrating a method of extracting heart rate response features from response data, as performed by processor 120 executing pre-processing module 154.

Method 700 begins with processor 120 receiving response data from a subject at step 320, as described above with reference to method 300 of FIG. 3. Specifically, step 320 of method 700 requires that the received response data is generated by detector optodes 164. To receive the response data, processor 120 may first instruct light output module 170 to cause optodes 162 to emit light by sending source optodes 162 signals via transmission channels 168. Detector optodes 164 may be caused to generate signals based on an amount of light captured, and to output the signals to data input module 180 via measurement channels 166.

Method 700 specifically relates to cases where signal elements caused by the heart rate of a subject have been kept for further analysis as described above. Processor 120 may be caused to execute method 700 to extract these from the response data.

According to some embodiments, at step 705 processor 120 executing pre-processing module 154 may first be caused to evaluate the signal quality of each channel from which response data was received. For example, where response data is received from detector optodes 164 via measurement channels 168, each measurement channel 168 may be evaluated for signal quality. This may be done by calculating a scalp coupling index (SCI) for each channel 168. As good skin contact between the optodes 162/164 and the scalp yields an observable fluctuation corresponding to cardiac pulsation, two channels from the same source-detector pair 162/164 are highly correlated due to the large cardiac signal if the scalp and optodes 162/164 are in good contact. To calculate the SCI, processor 120 may be caused to pass the signals of two channels 166 and 168 corresponding to a source/detector optode pair 162/164 through a bandpass filter in the cardiac frequency band. For example, the signals may be passed through a bandpass filter between 1.5 to 3.33 Hz when the subject is an infant, and between 0.5 and 2.0 Hz when the subject is an adult. According to some embodiments, the bandpass filter may be selected to be wide enough to be outside typical heart rate ranges for adults and infants.

Processor 120 may determine the SCI based on the correlation between the filtered signals. Processor 120 may use the determined SCI to reject channels that are determined to be of a poor signal quality, such as channels having a SCI of less than a predetermined threshold value. According to some embodiments, the predetermined threshold may be between 0.5 and 0.9, for example. According to some embodiments, the predetermined threshold may be around 0.8.

At step 710, processor 120 executing pre-processing module 154 may be caused to normalise the data signals. This may comprise passing the response data signals through a filter, which may be a bandpass filter in some embodiments. The bandpass filter may be selected to keep signal data likely to relate to heart rate data. For example, in some embodiments, processor 120 may be caused to pass the response data signals through a 1.50-3.33 Hz bandpass filter, to correspond to a heart rate range of between 90 and 200 bpm. This may be particularly suitable for use when testing infants. In some embodiments, processor 120 may be caused to pass the response data signals through a 0.50-2.0 Hz bandpass filter, which may be particularly suitable for use when testing adults. The filter may be an 8th order Butterworth filter in some embodiments. The resultant time-series signals may be normalised by dividing them with the envelope of the channel. This may be done using the default HILBERT function in MATLAB, for example, or another suitable envelope finding method.

At step 715, processor 120 executing pre-processing module 154 may be caused to de-multiplex the data to improve the data resolution. According to some embodiments, where a plurality of source and detector optodes 162/164 are used, the source optodes 162 may not turn on at the same time, but might instead be configured to turn on at individual times, to avoid detector readings from mixing up light from different source optodes. For example, the NIRScout device has a sampling rate of 62.5 Hz, but this 62.5 Hz sample rate is shared among all source optodes 162. Where eight source optodes 162 are used and are illuminated sequentially in a particular illumination pattern, the fNIRS sample rate per channel is actually 62.5 HZ/8, being 7.8125 Hz. However, while the data points are collected at individual times, with the data from one source optode 162 being (1/Fs=) 0.128 sec delayed relative to those from the source optode 162 that is switched on directly before it, some devices such as the NIRScout device are configured to store all the data points from one illumination sequence, where each of the eight sources in this example is turned on sequentially, onto a single timestamp.

The resolution of the collected data can be increased by using the correct time-offset for each source optode 162, according to the sequence of illumination, to correctly space out the readings from each source optode 162. As each source optode 162 contributes to four or six channels 166 (being 2 to 3 source-detector optode channels per wavelength), each timestamp still contains four to six data points. Processor 120 may be configured to average the data points per timestamp to produce one sample per timestamp. This method effectively increases the sampling frequency of the data by combining the data across all channels, producing a single waveform that represents the cardiac fluctuations. In this example, the original sampling frequency from the device of 62.5 Hz can be recovered.

At step 720, processor 120 executing pre-processing module 154 may be caused to smooth the waveform derived at step 715. This may be done using a 10-point moving average smoothing window. In some embodiments, a smoothing window with more or less than 10 points may be used, or a different smoothing method may be employed.

At step 725, processor 120 executing pre-processing module 154 may be caused to identify and correct identified bad regions in the waveform. During the testing process, the subject may move, causing transient artefacts that may disrupt the waveform in some of the measurement channels 168. Processor 120 may be configured to identify and remove these artefacts.

Processor 120 may do this by first identifying the noisy sections of the received data. In some embodiments, this may be done by using a sliding root-mean-square (RMS) window to calculate the signal power of the received data. For example, the RMS window may be around 50 samples long, or equivalent to around 0.8 sec. As noisy sections of data are likely to be more random compared to the sinusoidal shape of a clean heart rate waveform, processor 120 may be configured to determine that a low signal power corresponds to a noisy section of data, or a lack of heart rate information in the waveform. According to some embodiments, processor 120 may be configured to flag any point in the data where the calculated RMS drops below a first predetermined threshold value, which may be around 0.3, for example. The time region around the flagged point may be expanded until the RMS value exceeds a second predetermined threshold value that is higher than the first predetermined threshold value, which may be 0.4 in some embodiments. Processor 120 may be configured to perform an automated repair of the region by replacing it with a fitted sine function. The sine function may be selected as one that best estimates that region's peak Fourier power. If the fit has a mean squared error term of more than a predetermined value, the region may be deemed by processor 120 as not repaired and may be rejected. For example, the predetermined value may be 0.4 arbitrary units, in some embodiments. If the mean squared error term is less than the predetermined value, the sine wave is kept.

At optional step 730, processor 120 executing pre-processing module 154 may be caused to estimate an inter-beat interval (IBI) of the instantaneous heart rate based on the cleaned cardiac waveform. According to some embodiments, processor 120 may perform this by determining the peak of the waveform, and calculating the peak-to-peak distance, yielding the inter-beat interval. According to some alternative embodiments, processor 120 may instead estimate a heart rate using a zero-crossing estimation method, which may be less susceptible to random noise.

As cardiac waveforms are sinusoidal-like oscillations, processor 120 may determine the timing of each peak. In some embodiments, it does so by computing the midpoint of pairs of zero-crossings. In some embodiments, it does so by computing the time between zero-crossings of the same sign (i.e., positive-going or negative-going zero-crossings only). If one pair of zero-crossings is less than a predetermined threshold of milliseconds apart, processor 120 may be configured to reject the second zero-crossing in the pair. The threshold may be selected to reject zero-crossings that would correspond to a heart rate of over a predetermined value, such as 200 or 240 BPM. For example, according to some embodiments, zero-crossings that are determined to be less than 150 milliseconds apart may be rejected in some embodiments, as these are assumed to be noise. Processor 120 may be configured to then calculate the IBI by taking the time difference between two consecutive peaks. IBI values outside three standard deviations (SD) of the mean IBI may be rejected.

At step 735, processor 120 executing pre-processing module 154 may be caused to determine the instantaneous heart rate in beats-per-minute by dividing 60 by the calculated IBI. The plot of heart rate versus time may then be used to create the heart rate waveform. Processor 120 may also resample the heart-rate waveform, which would be timestamped to each beat's timing, in some embodiments, to an evenly spaced timing at 1000 Hz. This may be done using a tool such as the MATLAB® Resample function. The waveform may then be filtered, which may be done using a 0.02 to 0.2 Hz bandpass filter, in some embodiments.

At optional step 740, processor 120 executing pre-processing module 154 may be caused to perform epoch extraction to extract data in a predetermined time window with respect to the stimulus onset. For example, the epoch extraction may extract the data recorded between 3 seconds before and 20 seconds after the stimulus onset. Epochs may be baseline corrected by determining the mean of the response pre-stimulus, and subtracting this from the response data. According to some embodiments, the pre-stimulus response may be taken from 3 to 0 seconds before stimulus onset. According to some embodiments, each epoch may also be converted to a heart rate percentage change relative to this pre-stimulus region.

At optional step 745, to simplify each epoch for further analysis, processor 120 executing pre-processing module 154 may be caused to summarise each epoch to a single value by extracting the mean heart rate response within a time window, referred to as the response size. The time window may be defined as the period where the peak amplitude of the grand average response is reduced by half, for example.

The extracted measures determined at step 735 and/or step 745 may be used by processor 120 in executing steps 335 to 365 of method 300 to determine an output, as described in further detail above with reference to FIG. 3.

FIGS. 8 to 15 show particular examples of stimulation protocols and the results obtained from exposing subjects to those protocols. FIGS. 8 to 10 relate to a stimulation protocol as described above with reference to FIG. 4, and FIGS. 11 to 14 relate to a stimulation protocol as described above with reference to FIG. 5. FIG. shows how a stopping criteria would work for either stimulus presentation protocol.

FIG. 8 shows a diagram 800 illustrating a first stimulation protocol, as described above with reference to FIG. 4. The protocol comprises a plurality of stimulus blocks 810. In the illustrated embodiment, each stimulus block 810 comprises ten 500 msec speech stimuli 820. In alternative embodiments, each block may comprise any number of speech stimuli of any length. For example, each stimulus block 810 may comprise between 5 and 20 speech stimuli, where the speech stimuli are between 100 msec and 1 s long. Stimulus blocks 810 may be 1 to 15 seconds long. According to some embodiments, stimulus blocks 810 may be 4 to 6 seconds long. According to some embodiments, stimulus blocks 810 may be around 5 seconds long.

In the illustrated embodiments, each stimulus block 810 is separated by a 9 second silence interval 830. The silence intervals 830 may be 5 to 35 seconds long in some embodiments. According to some embodiments, the silence intervals may be 9 to 14 seconds long. According to some embodiments, the silence intervals may be 22 to 32 seconds long.

Each stimulus block 810 forms part of a stimulus condition, with each stimulus condition having five identical stimulus blocks 810. The stimulus conditions comprise a first habituation condition 840, a second habituation condition 850, and a novel or dishabituation condition 860.

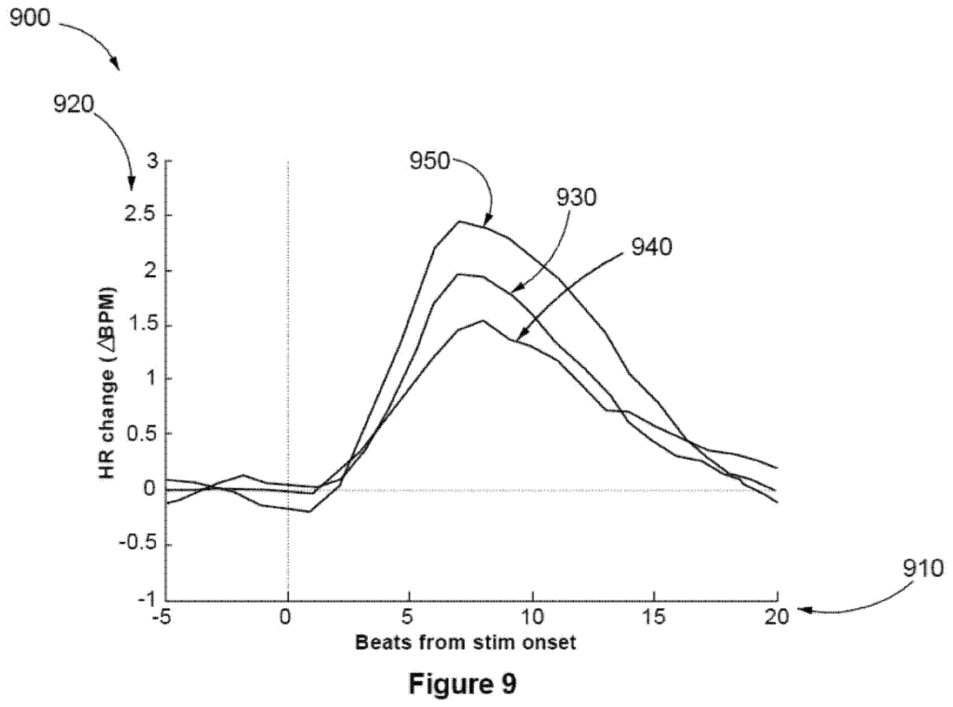

FIG. 9 shows a graph 900 of the results of presenting the first stimulation protocol as illustrated in FIG. 8 to a number of subjects in an experiment. In particular, FIG. 9 shows the heart rate response exhibited by the subjects.

In the study, auditory stimulation was provided to sleeping infants to evoke an fNIRS response. 23 infants aged between 2 and 10 months and that had passed screening tests were presented three consonant-vowel contrast pairs at 65 dB SPL bilaterally via insert earphones. The stimuli were presented using the habituation/dishabituation test paradigm as described above with reference to FIG. 8. Specifically, the test subjects in the study were presented with multiple runs of the stimuli while they remained asleep, meaning that each infant had a different total number of runs. In the trial, the number of runs ranged from 5 to 10.

In a first part of the experiment, one speech stimuli block 810 was presented repeatedly. This corresponds to the stimulus blocks as shown in FIG. 8 as the first habituation condition 840 and the second habituation condition 850. The heart rate of the subjects was measured, and was found to reduce in amplitude over time, demonstrating the habituation response. When a contrasting stimuli block was presented, as shown in FIG. 8 as the dishabituation condition 860, this stimulus was found to evoke a larger heart rate change response, demonstrating the dishabituation response. The difference of heart rate change response between the novel and habituation stimuli characterises the speech discrimination ability.

Graph 900 has an x-axis 910 showing the time in beats, from 5 beats before the stimulation onset at time 0 and over a 25 beat epoch. Graph 900 further includes a y-axis 920 showing the measured change in heart rate as a relative change in measured heart beats per minute. Response data 930 shows the response as measured while presenting a first habituation stimuli, such as that shown in FIG. 8 as the first habituation condition 840. This corresponds to a detection response, being a positive spike in heart rate, peaking at around 3-4 seconds (6-8 beats) after the onset of the stimulation. Response data 940 shows the response measured for a second habituation stimuli, comprising the same repeating sound as the first repeating stimuli, such as that shown in FIG. 8 as the second habituation condition 850. As seen from the graph, the heart rate response to the stimuli still has a positive peak, but this peak amplitude has decreased over time (compared to response data 930) as the subject is habituated to the sound. Response data 950 shows the response to a third stimuli, comprising different sounds to the first and second stimuli, such as that shown in FIG. 8 as the dishabituation condition 860. As seen from the graph, the heart rate response to the third, novel stimuli increased compared to the first and second stimuli to which the subject has been habituated. This increase in heart rate response amplitude to a novel sound shows that the subject has discriminated between the sound repeated in the first and second stimuli, and the new sound in the third stimuli. Heart rate data could be used in combination with neural response data to further increase the robustness of speech discrimination.

However, as the stimuli in this study was separated by a silence period, as indicated by silence intervals 830 in FIG. 8, both the habituated and non-habituated sounds in this study may have generated a detection response. This can make it more difficult to determine whether there also exists a discrimination response, as a statistically significant increase of the response amplitude to the dishabituation stimuli relative of the response to the habituation stimuli is necessary.

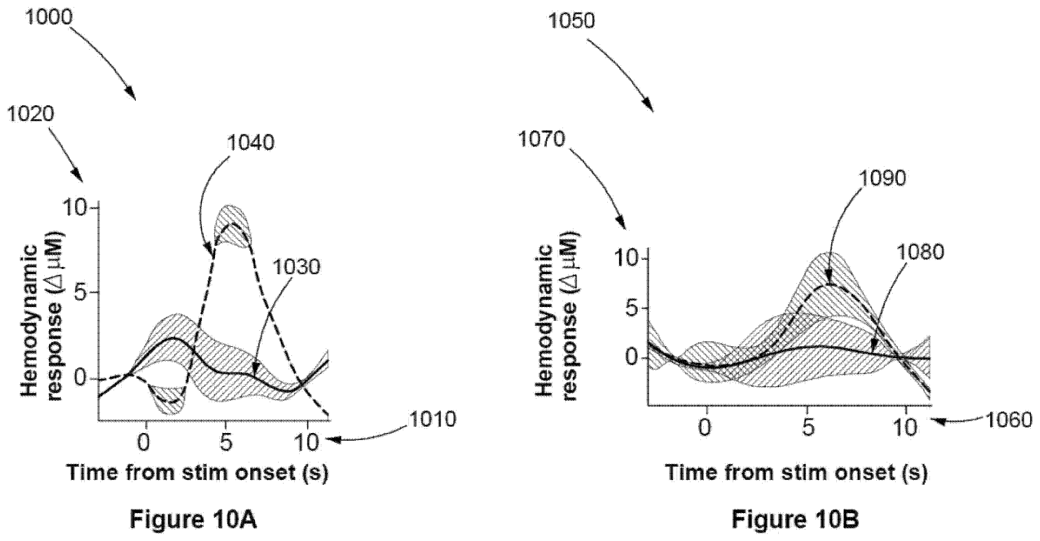

FIGS. 10A and 10B show further graphs of the results of presenting the first stimulation protocol as illustrated in FIG. 8 to one subject in an experiment. In particular, FIGS. 10A and 10B show the hemodynamic response exhibited by the subjects.

FIG. 10A shows a graph 1000 illustrating the mean detection hemodynamic response for one infant, as a change in measured HbO relative to a baseline, exhibited in response to all auditory stimuli in the experiment, as described above with reference to the first stimulus delivery protocol as shown in FIG. 8, particularly with reference to the stimulus blocks 810 in the periods 840, 850 and 860. The x-axis 1010 shows the time in seconds from a stimulation onset over a 14 second period, and the y-axis 1020 shows the measured change in hemodynamic (oxygenated hemoglobin) response as a change measured in μM. Response data 1030 shows the hemodynamic response to a control condition, which simulates the absence of a hearing response by randomising the stimulus trigger timing to not align with the stimulus onset, while response data 1040 shows the standard detection response to the onset of a sound. Response data 1040 shows a large detection response evoked by the stimulus by comparing the response to a baseline defined by the average of data over 3 s of silence before the stimulus onset. The control data (simulated absent-hearing response data 1030) was not significantly different from the baseline defined as the average of data from over 3 s before the stimulus onset, indicating that a false positive response is unlikely for this infant.

FIG. 10B shows a graph 1050 illustrating the subtraction of the baseline-corrected responses evoked by stimulus blocks 810 in the habituation period of 850 from the responses evoked by stimulus blocks 810 in the novel period 860 based on the first stimulation protocol as described above with reference to FIG. 8. The x-axis 1060 shows the time in seconds from the stimulation onset over a 14 second period, and the y-axis 1070 shows the difference in change in hemodynamic (oxygenated hemoglobin) response measured in μM. Data 1090 shows the differences described above. Discrimination is determined by comparing the data 1090 to the baseline calculated as the average of the data over 3 s before the stimulus onset. Data 1080 shows the difference between two sets of control responses that were created by randomising the timing of the stimulus triggers to not align with the stimulus onsets. The low probability of a false positive result for this infant was shown by determining that the difference data 1080 was not significantly different to the baseline.

FIG. 11 shows a diagrammatic representation 1100 of a second stimulation protocol, as described above with reference to FIGS. 5A and 5B. The second stimulation protocol commences at a time 1105, and comprises a detection period 1110 corresponding to method 500 of FIG. 5A, and a discrimination period 1120 corresponding to method 550 of FIG. 5B. Detection period 1110 may begin with a period of silence 1130, which may be around 5 minutes long, in some embodiments. According to some embodiments, detection period 1110 may begin without a silence period. Stimulus blocks 1140 are then presented, separated by silence intervals 1150. The silence intervals may be 5 to 35 seconds long in some embodiments. According to some embodiments, the silence intervals may be 9 to 14 seconds long. According to some embodiments, the silence intervals may be 22 to 32 seconds long. In the illustrated embodiment, a minimum silence interval of 22 s was used. Stimulus blocks 1140 may be 1 to 15 seconds long. According to some embodiments, stimulus blocks 1140 may be 4 to 6 seconds long. According to some embodiments, stimulus blocks 1140 may be around 5 seconds long.

According to some embodiments, stimulus blocks 1140 may comprise a repeated speech syllable or word. For example, in some embodiments, stimulus blocks 1140 may comprise the speech syllable “ba” repeated.

Detection period 1110 is used to confirm that the speech sounds that form stimulus blocks 1140 are audible to the subject before testing for speech discrimination, and to characterise the standard detection response for the individual subject. The stimuli in this period evoke the standard and expected detection response to any auditory stimulus in the auditory and prefrontal areas of the brain. The response is commonly described by a convolution of the stimulus boxcar function and the standard haemodynamic response, or, commonly for an auditory stimulus, having a positive peak HbO response around 6 seconds after the stimulus onset.

Discrimination period 1120 uses a non-silence baseline. In other words, novel stimulus blocks (containing novel sounds or a mixture of novel and standard sounds) are presented with a repeated standard sound between the blocks as the baseline. This means that when the novel sound was introduced, any response to the novel stimulus blocks must be due to the infant distinguishing between the novel and standard stimuli. This response arises due to the brain analysing the differences between the sounds, or performing discrimination between the two sounds. A response significantly different from the baseline (which, as described, may be determined based on data from the silence period before stimulus onset) determines a significant discrimination response.

Discrimination period 1120 begins with a habituation period 1160, comprising the non-silence baseline. For example, habituation period 1160 may comprise a repeated speech stimuli being played. According to some embodiments, habituation period 1160 may comprise the speech syllable “ba” being repeatedly played. According to some embodiments, habituation period 1160 may be around 5 minutes long. Stimulus blocks 1170 are then presented, separated by baseline intervals 1180.

Baseline intervals 1180 may comprise a repeated speech stimulus being played. Baseline intervals 1180 may comprise the same speech stimulus as that of stimulus blocks 1140. For example, baseline intervals 1180 may comprise the speech syllable “ba” being repeatedly played. Stimulus blocks 1170 may comprise a different repeated speech stimulus to that of baseline intervals 1180. According to some embodiments, the speech stimulus may differ between stimulus blocks 1170. For example, the speech stimulus to be repeatedly played during stimulation blocks 1170 may be randomly selected from a group of speech stimuli. According to some embodiments, the speech stimuli may include syllables or words such as “tea”, “bee”, “ga”, “pa”, and “ma”, for example. According to some embodiments, stimulus blocks 1170 may comprise a different repeated speech stimulus to that of baseline intervals 1180 alternating with the same speech stimulus as that of baseline intervals 1180. For example, a stimulus block 1170 may comprise alternating speech syllables “tea” and “ba”.

Baseline intervals 1180 may be 5 to 35 seconds long in some embodiments. According to some embodiments, baseline intervals 1180 may be 9 to 14 seconds long. According to some embodiments, baseline intervals 1180 may be 22 to 32 seconds long. Stimulus blocks 1170 may be 1 to 10 seconds long. According to some embodiments, stimulus blocks 1170 may be 4 to 6 seconds long. According to some embodiments, stimulus blocks 1170 may be 5 to 20 seconds long.

The discrimination period 1120 is used to test for discrimination between two different speech sounds. A significant response to the stimulus blocks 1170 implies the brain is processing differences between the novel sounds as presented during stimulus blocks 1170 and the standard sounds as presented during the baseline intervals 1180.

FIGS. 12A, 12B, 13A and 13B show graphs of the results of presenting the second stimulation protocol as illustrated in FIG. 11 to a group of subjects in an experiment. The subjects, 16 infants with normal hearing, were tested while sleeping. In particular, FIGS. 12A and 12B show the hemodynamic response exhibited by the subjects in the first phase of the protocol corresponding to detection period 1110 of FIG. 11, and FIGS. 13A and 13B show the hemodynamic response to the second phase corresponding to discrimination period 1120 of FIG. 11.

FIG. 12A shows a graph 1200 illustrating the average measured fNIRS neural responses to stimuli based on the first phase of the second stimulation protocol as described above with reference to FIG. 11, and corresponding to detection period 1110. The x-axis 1210 shows the time in seconds from the stimulation onset 1230 over a 25 second period, and the y-axis 1220 shows the measured change in neural response as a change in measured HbO relative to a baseline. Response data 1240 shows the detection response to the onset of a sound, as evoked by the stimulus blocks 1140 of detection period 1110 of the protocol shown in FIG. 11.

It can be seen that response data 1240 shows a large negative dip in HbO following the initial positive peak after the stimulus onset. The extended silence period of 22 s to 32 s used in this experiment after the offset of the sound as illustrated in FIG. 11 by silence interval 1150, compared to the silence period of 9 s used for the experiment that used in the first protocol as illustrated in FIG. 8 and the results of which are shown in FIG. 10, allows this negative dip to be visualized. According to some embodiments, a minimum time window of 20 s post-stimulus onset may allow the dip to be visualised. This time window may be a combination of a stimulus and a silence interval. For example, the time window may comprise a 2 s stimulus period followed by an 18 s silence interval; a 5 s stimulus period followed by an 15 s silence interval; a 10 s stimulus period followed by an 10 s silence interval; or any other combination of a stimulus period followed by a silence period where the duration of the stimulus period and the silence period is at least a total of 20 s. Research, such as that described in Lee et al., 2023 (Lee, O. W., Mao, D., Wunderlich, J., Balasubramanian, G., Haneman, M., Korneev, M., Mckay, C. M., 2023. Two independent response mechanisms to auditory stimuli measured with fNIRS in sleeping infants. PREPRINT. available at Research Square [https://doi.org/10.21203/rs.3.rs-2493723/v1]) has determined that the dip is probably caused by brain arousal in response to the auditory stimulus in the sleeping infant, and that the brain arousal response occurs concurrently with the standard auditory-system response to the sound, being the positive peak after stimulus onset.

FIG. 12B is a graph 1250 showing the result of applying a model to the results of FIG. 12A, in which the response 1240 was modelled to be a sum of two concurrent responses: the response 1280 of the auditory system to the sound (having a positive peak around 6 seconds after stimulus onset 1275) and the brain arousal response 1290 evoked by the sound (having a negative dip around 16 seconds after stimulus onset 1275). Such modelling may be performed by processor 120 performing step 345 of method 300, as described above.

The x-axis 1260 shows the time in seconds from the stimulation onset 1275 over a 25 second period, and the y-axis 1270 shows the modelled change in neural response as a change in measured HbO relative to a baseline. Response data 1280 shows the modelled auditory system response to the sound and response data 1290 shows the modelled brain arousal response. It was shown (Lee et al., 2023) that the auditory system response changes very little over repeated stimulus blocks, whereas the brain arousal response habituates with repeated presentations of the same stimulus.

FIG. 13A shows a graph 1300 illustrating the average measured fNIRS neural responses to stimuli based on the second phase of the second stimulation protocol as described above with reference to FIG. 11, and corresponding to discrimination period 1120. The x-axis 1310 shows the time in seconds from the stimulation onset 1330 over a 30 second period, and the y-axis 1320 shows the measured change in neural response as a change in measured HbO relative to a baseline. Response data 1335, 1340 and 1345 show the discrimination responses to three different phonemic contrasts, as evoked by the stimulus blocks 1170 of discrimination period 1120 of the protocol shown in FIG. 11. The three contrasts were “tea”, “ga” and “bee”, respectively, against a baseline of “ba”.

As in FIG. 12A, it can be seen that the response data 1335 and 1345 show both positive peaks and later negative dips.

FIG. 13B shows a graph 1350 in which the response data 1335, 1340 and 1345 of FIG. 13A have been separated into two components, being the average (positive) auditory system responses 1380, 1382 and 1384 and average (negative) brain arousal responses 1390, 1392 and 1394, respectively. Such modelling may be performed by processor 120 performing step 345 of method 300, as described above.

The x-axis 1360 shows the time in seconds from the stimulation onset 1375 over a 30 second period, and the y-axis 1370 shows measured change in neural response as a change in measured HbO relative to a baseline. In graph 1350, the shape of the brain arousal responses 1390, 1392 and 1394 were assumed to be the same as the modelled shape from the detection phase (FIG. 12B), with their amplitude determined by the peak negativity in response data 1335, 1340 and 1345.

In other embodiments, the two responses can be separated by using techniques such as Independent Component Analysis or Principal Component Analysis, using each epoch as an independent measuring source, or by using a modelling approach as described in (Lee et al., 2023).

It can be seen that when the brain arousal response is separated, the remaining auditory responses 1380, 1382 and 1384 are very similar for all three phonemic contrasts. However, the amplitude of the brain arousal response 1390, 1392 and 1394 differs among the phonemic contrasts, being largest for the contrast with the most different acoustic properties (“ba” versus “tea”) and smallest for the contrast with very subtle acoustic differences (“ba” versus “ga”). This sensitivity of the brain arousal response to differing degrees of acoustic contrast makes it a powerful tool to measure the degree of difficulty in discriminating the two sounds. In other words, there is a measure of whether the two sounds are discriminated or not, given by the positive acoustic system response 1380, 1382 and 1384, and a measure of how difficult the discrimination was to make, given by the amplitude of the brain arousal response 1390, 1392 and 1394.

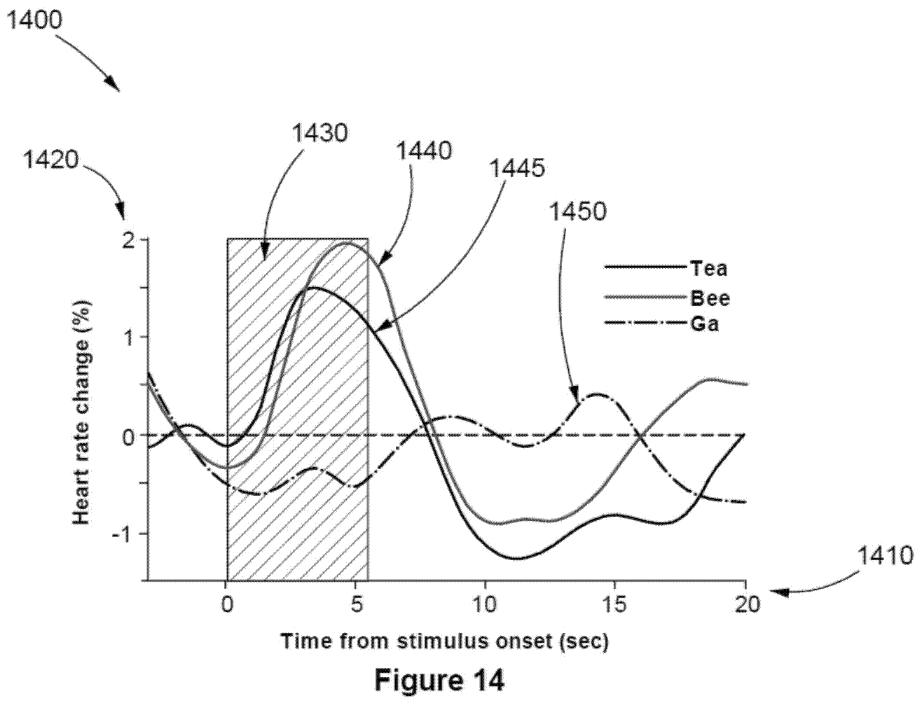

FIG. 14 shows a graph 1400 illustrating the average measured heart rate responses to discrimination stimuli for three different phonemic contrasts based on the second phase of the second stimulation protocol as described above with reference to FIG. 11, and corresponding to discrimination period 1120. In this graph, the heart rate change is averaged across infants for the first three instances of that contrast in the stimulation protocol.

The x-axis 1410 shows the time in seconds from the stimulation onset 1430 over a 23 second period, and the y-axis 1420 shows the measured change in heart rate response as a percentage change in measured heart rate relative to a baseline. Response data 1440, 1445 and 1450 show the discrimination responses as evoked by the stimulus blocks 1170 of discrimination period 1120 of the stimulus protocol shown in FIG. 11 based on three different phonemic contrasts, being “tea”, “bee”, and “ga”, respectively.

In the experiment, stimulus block 1170 contained one of three alternating speech syllables (“tea/ba”, “bee/ba” or “ga/ba”) with a baseline interval 1180 comprising the repeated speech syllable “ba”. While response data 1440 and 1445 show a heart rate response, response data 1450 shows that the heart rate response is absent in the contrast with the least acoustic difference (“ga/ba”). This difference of “ga/ba” from the stronger contrasts is analogous to the brain haemodynamic response associated with brain arousal shown in FIG. 13B. As in the detection phase of the experiment, the heart rate response adapts over the duration of the experiment.

FIG. 15 shows a graph 1500 illustrating the method of applying a stopping criterion to determine when to end the experiment procedure as described above with reference to step 580 of method 500, using data from one infant and the second protocol as referenced in FIG. 11. X-axis 1510 shows the number of trials completed in the experiment. Y-axis 1520 shows the entropy, as calculated from a statistical distribution of comparisons between performance statistics from a set of responses gathered from the current trial window in comparison to the previous trial. Data 1540 shows how the entropy calculation changes as more data from additional trials are obtained. A stopping point is determined by the entropy falling below a criterion value or stopping threshold 1530. In this example, the criterion entropy value was 1, and the experiment could be stopped after 12 trials. In other embodiments, the entropy criterion can be between 0 and 2, or any criterion that represents stationarity of the distribution.

Based on the outcomes of the experiments described above with reference to FIGS. 9, 10A, 10B, 12A, 12B, 13A, 13B, 14 and 15, it is possible to assess the discrimination ability of a subject by measuring physiological signals in response to habituation and dishabituation stimuli.

According to some embodiments, the system and methods described above can be used in combination with other physiological response signals, such as EEG measures of electrical brain responses to auditory stimulation, using standard methods such as ABR (auditory brainstem response), CAEP (Cortical auditory evoked potentials) and ASSR (auditory steady-state responses). The simultaneous use of multi-dimensional data that includes both fNIRS and/or biosignal data along with EEG data may optimise the accuracy and/or reliability of the measurements.

According to some embodiments, the methods described above may be used in combination with other objective measures of hearing, such as EEG, physiological responses such as skin conductance, respiration rate, blood pressure changes, and with any available behavioural measures or observations.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of assessing the hearing of a subject, the method comprising:
presenting a first aural stimulus to the subject;
presenting a second aural stimulus to the subject, the second aural stimulus being different to the first aural stimulus;
receiving at least one physiological response signal relating to the second aural stimulus received by the subject;
comparing at least one parameter related to the at least one physiological response signal with a control parameter; and
determining an auditory discrimination response of the subject based on the outcome of the comparison.

2. The method of claim 1, wherein the first aural stimulus and the second aural stimulus are presented consecutively without any silence interval between the first aural stimulus and the second aural stimulus.

3. The method of claim 1 or claim 2, further comprising modelling the physiological response signal to create a response model.

4. The method of claim 3, wherein the model captures unique statistical properties in the neural response data pertaining to its neighbourhood covariance structure relative to aural stimulation onset, spanning the length of the expected response and across one or more responses.

5. The method of claim 3 or claim 4, wherein the model is generated using a stochastic process.

6. The method of any one of claims 3 to 5, wherein the response model captures at least two concurrent neural responses in response to one aural stimulus.

7. The method of claim 6, wherein the at least one of the neural processes relates to activation of the cortical auditory system.

8. The method of claim 7, further comprising using the modelled neural response data relating to the activation of the cortical auditory system to determine a measure corresponding to whether or not the subject discriminated between the first aural stimulus and the second aural stimulus.

9. The method of any one of claims 6 to 8, wherein at least one of the neural processes relates to activation of the brain arousal system.

10. The method of claim 9, further comprising using the modelled neural response data relating to the activation of the brain arousal system to determine a measure corresponding to a difficulty that the subject experienced in discriminating between the first aural stimulus and the second aural stimulus.

11. The method of any one of claims 3 to 10, wherein the control parameter is determined based on a model of a baseline response signal.

12. The method of claim 11, wherein the baseline response signal comprises a physiological response signal measured during a time not aligned with a time when the second aural stimulus is being delivered to the subject.

13. The method of claim 12, wherein the baseline response signal comprises a physiological response signal measured during a time when the first aural stimulus is being delivered to the subject.

14. The method of claim 12, wherein the baseline response signal comprises a physiological response signal measured during a time when no stimulus is being delivered to the subject.

15. The method of any one of claims 11 to 14, wherein comparing at least one parameter related to the at least one physiological response signal with a control parameter comprises using test statistics to compare whether the response model and the model of the baseline response signal are different.

16. The method of any one of claims 3 to 15, where the data used to generate the models are permuted and create a collection of statistical measures.

17. The method of claim 16, where the collection of statistical measures is used to generate a confidence level for the response detection decision.

18. The method of any one of claims 1 to 17, wherein the first aural stimulus comprises a repeating speech syllable presented as a habituation stimulus.

19. The method of any one of claims 1 to 18, wherein the second aural stimulus comprises a repeating speech syllable presented as a dishabituation stimulus.

20. The method of any one of claims 1 to 19, wherein the physiological response signal comprises fNIRS data generated by at least one optode located on a scalp of the subject.

21. The method of claim 20, wherein the fNIRS data comprises neural response data.

22. The method of claim 20 or claim 21, wherein the fNIRS data comprises cardiac data.

23. The method of any one of claims 1 to 19, wherein the physiological response signal comprises cardiac data generated by a cardiac monitor.

24. The method of claim 20 or claim 23, wherein the cardiac data comprises at least one of a heart rate, heart rate variability, blood pressure and/or breathing rate.

25. The method of any one of claims 1 to 24, wherein the first aural stimulus and the second aural stimulus are selected to create a speech contrast, the speech contrast being the result of a difference in one or more speech features between the first aural stimulus and the second aural stimulus for which a discrimination assessment is desired.

26. The method of claim 25, wherein the speech contrast comprises at least one of a vowel contrast, consonant contrast, vowel difference cue, or a difference in place of consonant articulation.

27. The method of any one of claims 1 to 26, further comprising determining an auditory discrimination response of the subject based on the outcome of the comparison.

28. The method of any one of claims 1 to 27, wherein the first aural stimulus is presented for a duration of between 1 and 35 seconds.

29. The method of claim 28, wherein the first aural stimulus is presented for a duration of between 9 and 14 seconds.

30. The method of any one of claims 1 to 29, wherein the second aural stimulus is presented for a duration of between 1 and 35 seconds.

31. The method of claim 30, wherein the second aural stimulus is presented for a duration of less than 10 seconds.

32. The method of any one of claims 1 to 31, further comprising repeating the steps of presenting the first aural stimulus, presenting a second aural stimulus and receiving at least one physiological response signal.

33. The method of claim 32, wherein the steps of presenting the first aural stimulus, presenting a second aural stimulus and receiving at least one physiological response signal are repeated until a stopping criterion is met.

34. The method of claim 33, wherein the collection of statistical measures is used to calculate a stopping criterion.

35. The method of any one of claims 32 to 34, further comprising:
combining the received physiological response signals into a combined single physiological response signal, and
combining at least two physiological response signals into a combined single baseline response signal,
wherein comparing the at least one parameter related to the at least one physiological response signal with a control parameter comprises comparing the combined single physiological response signal with the combined single baseline response signal.

36. The method of claim 35, wherein combining at least two physiological response signals into a combined single baseline response signal comprises combining the at least two physiological response signals measured during a time not aligned with a time when the second aural stimulus is being delivered to the subject.

37. The method of claim 35 or claim 36, wherein combining at least two physiological response signals into a combined single baseline response signal comprises combining the at least two physiological response signals measured during a time when no stimulus is being delivered to the subject.

38. The method of any one of claims 35 to 37, wherein combining at least two physiological response signals into a combined single baseline response signal comprises combining the at least two physiological response signals measured during a time when the first aural stimulus is being delivered to the subject.

39. The method of any one of claims 1 to 38, further comprising communicating the outcome to a user via a user interface.

40. The method of any one of claims 1 to 39, further comprising communicating the outcome to an external computing system.

41. A system for assessing the hearing of a subject, the system comprising:
at least one stimulation delivery member;
at least one physiological signal sensor;
memory storing executable code; and
a processor configured to access and execute the code stored in the memory;
wherein, when executing the code, the processor is caused to:
present, via the at least one stimulation delivery member, a first aural stimulus to the subject;
present, via the at least one stimulation delivery member, a second aural stimulus to the subject, the second aural stimulus being different to the first aural stimulus;
receive, from the at least one physiological signal sensor, at least one physiological response signal relating to the second aural stimulus received by the subject;
compare at least one parameter related to the at least one physiological response signal with a control parameter; and
determine an auditory discrimination response of the subject based on the outcome of the comparison.
